SEQUENCE LISTING (4 sequences)

SEQ ID NO: 1 - protein, 406 aa, Homo sapiens
MVPPPPSRGGAARGQLGRSLGPLLLLLALGHTWTYREEPEDGDREICSESKIATTKYPCLKSSGELTTCYRKKCCKGYKFVLGQCIPEDYDVCAEAPCEQQCTDNFGRVLCTCYPGYRYDRERHRKREKPYCLDIDECASSNGTLCAHICINTLGSYRCECREGYIREDDGKTCTRGDKYPNDTGHEKSENMVKAGTCCATCKEFYQMKQTVLQLKQKIALLPNNAADLGKYITGDKVLASNTYLPGPPGLPGGQGPPGSPGPKGSPGFPGMPGPPGQPGPRGSMGPMGPSPDLSHIKQGRRGPVGPPGAPGRDGSKGERGAPGPRGSPGPPGSFDFLLLMLADIRNDITELQEKVFGHRTHSSAEEFPLPQEFPSYPEAMDLGSGDDHPRRTETRDLRAPRDFYP

SEQ ID NO: 2 - protein, 419 aa, Homo sapiens
MHLLGFFSVACSLLAAALLPGPREAPAAAAAFESGLDLSDAEPDAGEATAYASKDLEEQLRSVSSVDELMTVLYPEYWKMYKCQLRKGGWQHNREQANLNSRTEETIKFAAAHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRRSLPATLPQCQAANKTCPTNYMWNNHICRCLAQEDFMFSSDAGDDSTDGFHDICGPNKELDEETCQCVCRAGLRPASCGPHKELDRNSCQCVCKNKLFPSQCGANREFDENTCQCVCKRTCPRNQPLNPGKCACECTESPQKCLLKGKKFHHQTCSCYRRPCTNRQKACEPGFSYSEEVCRCVPSYWKRPQMS

SEQ ID NO: 3 - DNA, 2076 bp, Homo sapiens
cggggaaggggagggaggagggggacgagggctctggcgggtttggaggggctgaacatcgcggggtgttctggtgtcccccgccccgcctctccaaaaagctacaccgacgcggaccgcggcggcgtcctccctcgccctcgcttcacctcgcgggctccgaatgcggggagctcggatgtccggtttcctgtgaggcttttacctgacacccgccgcctttccccggcactggctgggagggcgccctgcaaagttgggaacgcggagccccggacccgctcccgccgcctccggctcgcccagggggggtcgccgggaggagcccgggggagagggaccaggaggggcccgcggcctcgcaggggcgcccgcgcccccacccctgcccccgccagcggaccggtcccccacccccggtccttccaccatgcacttgctgggcttcttctctgtggcgtgttctctgctcgccgctgcgctgctcccgggtcctcgcgaggcgcccgccgccgccgccgccttcgagtccggactcgacctctcggacgcggagcccgacgcgggcgaggccacggcttatgcaagcaaagatctggaggagcagttacggtctgtgtccagtgtagatgaactcatgactgtactctacccagaatattggaaaatgtacaagtgtcagctaaggaaaggaggctggcaacataacagagaacaggccaacctcaactcaaggacagaagagactataaaatttgctgcagcacattataatacagagatcttgaaaagtattgataatgagtggagaaagactcaatgcatgccacgggaggtgtgtatagatgtggggaaggagtttggagtcgcgacaaacaccttctttaaacctccatgtgtgtccgtctacagatgtgggggttgctgcaatagtgaggggctgcagtgcatgaacaccagcacgagctacctcagcaagacgttatttgaaattacagtgcctctctctcaaggccccaaaccagtaacaatcagttttgccaatcacacttcctgccgatgcatgtctaaactggatgtttacagacaagttcattccattattagacgttccctgccagcaacactaccacagtgtcaggcagcgaacaagacctgccccaccaattacatgtggaataatcacatctgcagatgcctggctcaggaagattttatgttttcctcggatgctggagatgactcaacagatggattccatgacatctgtggaccaaacaaggagctggatgaagagacctgtcagtgtgtctgcagagcggggcttcggcctgccagctgtggaccccacaaagaactagacagaaactcatgccagtgtgtctgtaaaaacaaactcttccccagccaatgtggggccaaccgagaatttgatgaaaacacatgccagtgtgtatgtaaaagaacctgccccagaaatcaacccctaaatcctggaaaatgtgcctgtgaatgtacagaaagtccacagaaatgcttgttaaaaggaaagaagttccaccaccaaacatgcagctgttacagacggccatgtacgaaccgccagaaggcttgtgagccaggattttcatatagtgaagaagtgtgtcgttgtgtcccttcatattggaaaagaccacaaatgagctaagattgtactgttttccagttcatcgattttctattatggaaaactgtgttgccacagtagaactgtctgtgaacagagagacccttgtgggtccatgctaacaaagacaaaagtctgtctttcctgaaccatgtggataactttacagaaatggactggagctcatctgcaaaaggcctcttgtaaagactggttttctgccaatgaccaaacagccaagattttcctcttgtgatttctttaaaagaatgactatataatttatttccactaaaaatattgtttctgcattcatttttatagcaacaacaattggtaaaactcactgtgatcaatatttttatatcatgcaaaatatgtttaaaataaaatgaaaattgtattat

SEQ ID NO: 4 - protein, 354 aa, Homo sapiens
MYREWVVVNVFMMLYVQLVQGSSNEHGPVKRSSQSTLERSEQQIRAASSLEELLRITHSEDWKLWRCRLRLKSFTSMDSRSASHRSTRFAATFYDIETLKVIDEEWQRTQCSPRETCVEVASELGKSTNTFFKPPCVNVFRCGGCCNEESLICMNTSTSYISKQLFEISVPLTSVPELVPVKVANHTGCKCLPTAPRHPYSIIRRSIQIPEEDRCSHSKKLCPIDMLWDSNKCKCVLQEENPLAGTEDHSHLQEPALCGPHMMFDEDRCECVCKTPCPKDLIQHPKNCSCFECKESLETCCQKHKLFHPDTCSCEDRCPFHTRPCASGKTACAKHCRFPKEKRAAQGPHSRKNP

(19) United States
(12) Patent Application Publication
(10) Pub. No.: US 2016/0331807 A1
(43) Pub. Date: Nov. 17, 2016

(54) THERAPEUTIC USE OF VEGFR-3 LIGANDS

(71) Applicant: UNIVERSITY OF HELSINKI, Helsinki (FI)

(72) Inventors: Kari ALITALO, Helsinki (FI); Aleksanteri ASPELUND, Helsinki (FI); Tuomas TAMMELA, Boston, MA (US); Ilkka IMMONEN, Kauniainen (FI)

(73) Assignee: UNIVERSITY OF HELSINKI, Helsinki (FI)

(21) Appl. No.: 15/112,858

(22) PCT Filed: Jan. 20, 2015
(86) PCT No.: PCT/FI2015/050028

§ 371 Date: Jul. 20, 2016

(30) Foreign Application Priority Data
Jan. 21, 2014 (FI) 20145053

(51) Int. Cl. A61K 38/17; C07K 14/71

(52) U.S. Cl. CPC A61K 38/179; C07K 14/71

(57) ABSTRACT

The present invention relates to therapeutic methods, uses and compositions for treating glaucoma or ocular hypertension. More specifically, the present invention relates to methods, uses and compositions utilizing VEGFR-3 activating ligand VEGF-C.

### FIELD OF THE INVENTION

The present invention relates to therapeutic methods, uses and compositions for treating glaucoma or ocular hypertension. More specifically, the present invention relates to methods, uses and compositions utilizing VEGFR-3 activating ligand VEGF-C.

### BACKGROUND OF THE INVENTION

Glaucoma is a group of heterogeneous diseases characterized by chronic, degenerative optic neuropathy in which loss of axons and supporting structures leads to a characteristic excavation of the optic nerve head with resultant loss of visual field^{1,2}. Glaucoma is the second leading cause of blindness in the world³, affecting approximately 2.65% of the population over 40 years of age worldwide with increasing prevalence⁴. The most important, and the only modifiable risk factor for glaucoma is elevated intraocular pressure (IOP)¹. In accordance, patients suffering from ocular hypertension, defined as intraocular pressure higher than normal in the absence of optic nerve damage or visual field loss, are at risk for developing glaucoma.

IOP is determined by the balance between the rate of production and rate of removal of the aqueous humor (AH). AH is constantly produced by the ciliary epithelium, and the majority (70-90%) of the AH is removed by the trabecular outflow pathway. In this pathway, AH is sieved through the trabecular meshwork (TM), taken up by the Schlemm's canal (SC), and drained into episcleral veins via the aqueous veins (AV)^{1,5}. In glaucoma, the rate of fluid removal declines so that it no longer keeps pace with the rate of fluid formation, resulting in increased IOP and subsequent optic neuropathy^{3,6,7}. Randomized clinical trials have shown that reducing intraocular pressure slows the onset and progression of glaucoma, even in normotensive glaucoma^{8,9}. Therefore, current treatments of glaucoma are aimed at enhancing aqueous outflow by pharmacological or surgical means. However, in spite of the therapies available, normalization of IOP and arrest of glaucoma development is often not achieved. Moreover, current medical therapies require regular daily administration, rendering their efficacy dependent on patient compliance.

The Schlemm's canal (SC) is a unique ring shaped, endothelium-lined vessel that encircles the cornea¹⁰. It is the final barrier for the AH to cross before returning to systemic circulation⁵. Interestingly, patients with glaucoma have a smaller SC¹¹ and agenesis or hypoplasia of the SC has been implicated in primary congenital glaucomas¹²⁻¹⁴.

All current treatments of ocular hypertension and glaucoma are aimed at enhancing aqueous outflow by medical or surgical means. However, there is an unmet clinical need for new glaucoma therapies as current glaucoma treatment is broad and nonspecific due to the lack of understanding of the mechanisms by which aqueous outflow is regulated. Therefore, patients with glaucoma can continue to have loss of vision despite reductions of eye pressure.

For example, the treatment for uncontrolled glaucoma, trabeculotomy, often fails due to the development of fibrosis in the conjunctiva and episclera because of progressive fibroblast proliferation and collagen deposition at the site of the filtration bleb. This frequently leads to poor postoperative intraocular pressure control with subsequent progressive optic nerve damage. The use of adjunctive antifibrotic agents such as 5-fluorouracil (5-FU) and mitomycin C (MMC) has significantly improved the success rate of filtration surgery. However, because of their nonspecific mechanisms of action, these agents cause widespread cell death and apoptosis, resulting in potentially sight-threatening complications such as severe postoperative hypotony, bleb leaks, and endophthalmitis. Thus, alternative strategies are needed to prevent this from happening.

### BRIEF DESCRIPTION OF THE INVENTION

An object of the present invention is thus to provide specific methods and compositions for treating ocular hypertension or glaucoma. The purpose is to develop glaucoma therapies by stimulation of SC endothelial cells for therapeutic manipulation in order to decrease intraocular pressure or to enhance the intraocular pressure lowering effect of other glaucoma therapies.

The invention is based on the realization that VEGFR-3 stimulation with VEGF-C or any derivatives (hereafter VEGFR-3 ligands), can be used for stimulating the SC endothelium and/or therapeutically growing the SC to facilitate aqueous humor outflow. According to the invention VEGFR-3 ligands can be used either alone or in combination with other therapeutically effective agents and/or glaucoma surgery.

Advantages of the arrangements of the invention are that patients suffering from glaucoma or ocular hypertension may receive specific treatments, which are effective, safe and have as few side effects as possible. Also, by the methods and uses of the present invention, it is possible to combine other glaucoma treatments with manipulation of the SC.

The objects of the invention are achieved by a method and an arrangement, which are characterized by what is stated in the independent claims. The specific embodiments of the invention are disclosed in the dependent claims.

In one aspect, the present invention relates to a VEGFR-3 activating ligand or a composition comprising a VEGFR-3 activating ligand for use in treating ocular hypertension or glaucoma in a subject, wherein the VEGFR-3 activating ligand is VEGF-C.

In another aspect, the present invention relates to a method of treating ocular hypertension or glaucoma by administering to a subject in need thereof a VEGFR-3 activating ligand or a composition comprising a VEGFR-3 activating ligand, wherein the VEGFR-3 activating ligand is VEGF-C.

Further aspects of the present invention relate to enhancing surgical or pharmacological ocular hypertension or glaucoma treatments with a composition comprising VEGFR-3 ligand VEGF-C.

Further aspects of the present invention relate to use of VEGF-C or a composition comprising VEGF-C for the manufacture of a medicament for treatment of ocular hypertension or glaucoma in a subject.

Other aspects, specific embodiments, objects, details and advantages of the invention are set forth in the following drawings, detailed description and examples.

### BRIEF DESCRIPTION OF THE DRAWINGS

In the following the invention will be described in greater detail by means of preferred embodiments with reference to the attached drawings, in which

FIG. 1 demonstrates that the Schlemm's canal lining has a molecular identity of lymphatic endothelium. (a-m) Whole mount immunofluorescence staining of the adult murine eye using antibodies against PECAM-1, Prox1, and VEGFR-3. The entire thickness of the limbus is imaged by confocal imaging and the projections of subsets showing the Schlemm's canal (SC)(a-d), aqueous vein (AV)(e-h) and episcleral (ES) vasculature (i-l). The joining point of the AV into the SC is indicated by arrow and joining point of the AV into ES vein is indicated by arrowhead (e, h, i, l and m). * denotes lymphatic vessel, a artery, v vein and c capillary. (m) Visualization of the aqueous humor drainage route in a 90-degree y-axis projection of the above (A-L) confocal stack. CC denotes choriocapillaries. xyz-axis for orientation between (A-L) and (M). Immunofluorescence of the murine SC using antibodies against CCL21 (n) and LYVE-1 (o). (p) Visualization of the SC (dashed line) and episcleral lymphatic vessels (*) in vivo in Prox1-CreER^{T2}; R26-flox-STOP-flox-tdTomato reporter mice after administration of 4-OH-tamoxifen. (q-r) Immunohistochemical staining of Prox1 in the SC of human eye with negative control. (r) Visualization of the Zebrafish SC in immunofluorescence staining with antibodies against human Prox1 and mouse Prox1. Scale bars: 100 μm (A-L), 50 μm (M, S), and 200 μm (N-O, Q-R).

FIG. 2 visualizes that the Schlemm's canal develops postnatally from transscleral veins. (a-t) Visualization of the SC development by LSCM in whole mount immunofluorescence stained tissues. Antibodies against PECAM-1 (green), Prox1 (red), and VEGFR-3 (blue) were used. (u-y) Schematic drawing of the SC developmental stages. In (a-t), the entire thickness of the limbal vasculature was sectioned by LSCM. The subset of the confocal z-stacks selected for the immunofluorescence images is indicated by the dashed line in (u-y). Visualization of the entire stack with choriocapillaries (CC) and episcleral veins (EV) is shown in 3D volume renderings in the Supplementary Videos 1-5. Five developmental stages at P0, P1, P2, P4 and P7 were discerned. (a-d, u) P0: lateral sprouting (indicated by asterisks, inset in A) of transcleral veins toward adjacent transcleral veins. (e-h, v) P1: connection of adjacent transcleral veins by strings of future SC endothelium. (i-l, w) P2: Maturation and induction of Prox1 expression (indicated by arrow) (m-p, x) P4: Luminalization and strong expression of Prox1, induction of VEGFR-3 (indicated by arrowheads), regression of connections to CC's (indicated by hashtag), lateral sprouting (indicated by asterisks). (q-t, y) P7: Mature SC. Note that aqueous veins (indicated by arrow) do not regress. Scale bars: 200 μm (a-t), 50 μm (inset in a)

FIG. 3 shows that soluble VEGFR-3 and targeted deletion of VEGF-C inhibits normal SC development. (a-b) Analysis of SC morphology in transgenic K14-VEGFR-3(1-3)-Ig (R3(1-3)-Ig, n=3) and their wild type littermates (n=3) at P7, and in K14-VEGFR-3(4-7)-Ig (R3(4-7)-Ig, n=4) at P7. Immunofluorescence staining of the SC with antibodies against PECAM-1 (green) and Prox1 (red) (a) and quantitative analysis of SC surface area from one litter (c). (c-d) Analysis of changes in SC morphology in Vegfc^{flox/flox}; Rosa26-iCreER^{T2} (VEGF-C^{iΔR26}, n=4) and Vegfc^{flox/flox} littermates (control, n=5) at P7 after induction of Cre activity from P1 onwards with daily 4-OH-tamoxifen injections until P5. Immunofluorescence staining of the SC using antibodies against PECAM-1 (green) and Prox1 (red) (c), and quantitative analysis of SC surface area from two litters (f). (e-f) Analysis of SC morphology in VEGF-D^{-/-} (n=6) and wild type (n=3) littermates. Immunofluorescence staining of the SC with antibodies against PECAM-1 (green) and Prox1 (red) (e) and quantitative analysis of the SC surface area (f). Data represent mean±s.d. surface area in 0.181-mm² limbal areas. *P<0.05, **P<0.01, one-way ANOVA with Tukey's post-hoc test (b) or two-sample (unpaired Student's) two-sided t test assuming equal variance (d, f). Scale bars: 200 μm.

FIG. 4 demonstrates that VEGFR-2-function-blocking antibodies and targeted deletion of Vegfr3 in SC endothelium inhibit normal SC development. (a-b) Analysis of changes in SC morphology after injection of anti-VEGFR-3 antibodies (α-R3, n=4), anti-VEGFR-2 antibodies (α-R2, n=4), VEGFR-3 and VEGFR-2 antibodies in combination (α-R3+2, n=3) or control rat IgG (IgG, n=3) once daily during P0-P7 into littermate mice. Immunofluorescence staining of the SC using antibodies against PECAM-1 (green) and Prox1 (red) (a) and quantitative analysis of the PECAM-1-positive SC surface area from one litter (b). (c-d) Analysis of changes in the SC morphology in Vegfr3^{flox/flox}; Prox1-CreER^{T2} (R3^{iΔLEC}, n=3) and Vegfr3^{flox/flox} (control, n=3) mice at P7 after induction of Cre activity from P1 onwards with daily 4-OH-tamoxifen injections. Immunofluoresence staining of the SC using antibodies against PECAM-1 (green), Prox1 (red) and VEGFR-3 (blue), validation of VEGFR-3 deletion using VEGFR-3 staining (blue) (G, I) and quantitative analysis of PECAM-1-positive SC area from one litter (d). (e-f) Analysis of changes in the SC morphology in Vegfr2^{flox/flox}; Prox1-CreER^{T2} (R2^{iΔLEC}, n=4) and Vegfr2^{flox/flox} (control, n=4) mice at P7 after induction of Cre activity from P1 onwards with daily 4-OH-tamoxifen injections. Immunofluoresence staining of the SC using antibodies against PECAM-1 (green), Prox1 (red) and VEGFR-2 (blue), validation of VEGFR-2 deletion using VEGFR-2 staining (blue) (e) and quantitative analysis of PECAM-1-positive SC area from data pooled from two litters (f). Scale bars, 100 μm (a, c and d). Data represent mean±s.d. surface area in 0.181-mm² limbal area in mm². *P<0.05, **P<0.01, one-way ANOVA with Tukey's post-hoc test (b) or two-sample (unpaired Student's) two-sided t test assuming equal variance (d, f).

FIG. 5 shows that overexpression of VEGF-C induces sprouting, proliferation and migration of the SC EC's. Analysis of the changes in SC morphology and proliferation as well as in intraocular pressure after overexpression of VEGF-C, VEGF165 or a control. To identify proliferating ECs, the mice were injected with 100 mg/kg BrdU 2 h prior to sacrifice. (a-e) Adenoviruses encoding full-length VEGF-C (AdVEGF-C), VEGF165 (AdVEGF-A) or an “empty” CMV promoter (AdControl) were injected into the anterior chamber. (a) Immunofluorescence staining of the SC with antibodies against PECAM-1 (green), BrdU (red) and Prox1 (blue) at day 4 and day 14 after injection. Asterisk denotes sprouts of the SC endothelium. Illustration of the changes in limbal vascular anatomy after injection at day 14. Quantitative analysis of SC surface area (b) and sprouts extending toward the cornea or the sclera (c) in AdVEGF-C and AdControl treated eyes. Relative changes in intraocular pressure to baseline after injection at day 4 (b) and 14 (k). (f) Adeno-associated viruses encoding full-length VEGF-C or HSA were injected into the anterior chamber. Immunofluorescence staining of the SC with antibodies against PECAM-1 (green), BrdU (red) and Prox1 (blue) at week 6 after transduction and illustration of the changes in limbal vascular anatomy after injection at week 6 after injection. C, cornea, S, sclera, AC, anterior chamber, PC, posterior chamber. Data represent mean±s.d. Each dot represents data from one eye. Quantitative data represent mean from a 0.181-mm² limbal area. *P<0.05, **P<0.01, ***P<0.001, ****P<0.0001, two-sample (unpaired Student's) two-sided t test assuming equal variance (b) or one-way ANOVA with Tukey's post-hoc test (c-e).

FIG. 6 demonstrates that a single injection recombinant VEGF-C induces sprouting, proliferation and enlargement of the SCEC's associated with a sustained decrease in intraocular pressure. (a-b) Analysis of changes in SC morphology on day 4 after injection of recombinant VEGF-C, VEGF-165 or HSA into the anterior chamber on three consecutive days. To identify proliferating ECs, the mice were injected with 100 mg/kg BrdU 2 h prior to sacrifice. (a) Immunofluorescence staining of the SC with antibodies against PECAM-1 (green), BrdU (red) and Prox1 (blue) and illustration of the changes in limbal vascular anatomy after injections. Asterisks denotes sprouts of the SC endothelium. (b) Quantitative analysis of mean sprouts per field per eye toward the cornea or the sclera. Each dot represents data from one eye. (c-g) Analysis of effects of a single injection or recombinant VEGF-C (rVEGF-C) or mouse serum albumin (rMSA). (c) Mean relative change in IOP to baseline per eye on day 8, 11 and 14 after single injection. Day 8: −30.03%±5,599 vs. −6,684%±3,597. Day 11: −28.52%±5,034 vs. −5,198±3,106. Day 14: −27.86%±5,117 vs. −1,705%±4,625). (d) Representative macroscopic images of eyes on day 9 after injection (e) Immunofluorescence staining of the SC (e) and corneal/episcleral vasculature on day 14 after injection with antibodies against PECAM-1 (green), Prox1 (red) and VEGFR-3. (g) Representative images of H&E stained paraffin embedded sections of the eyes on day 14. *P<0.05, **P<0.01, ****P<0.0001, one-way ANOVA with Tukey's post-hoc test (b) or two-sample (unpaired Student's) two-sided t test assuming equal variance (c).

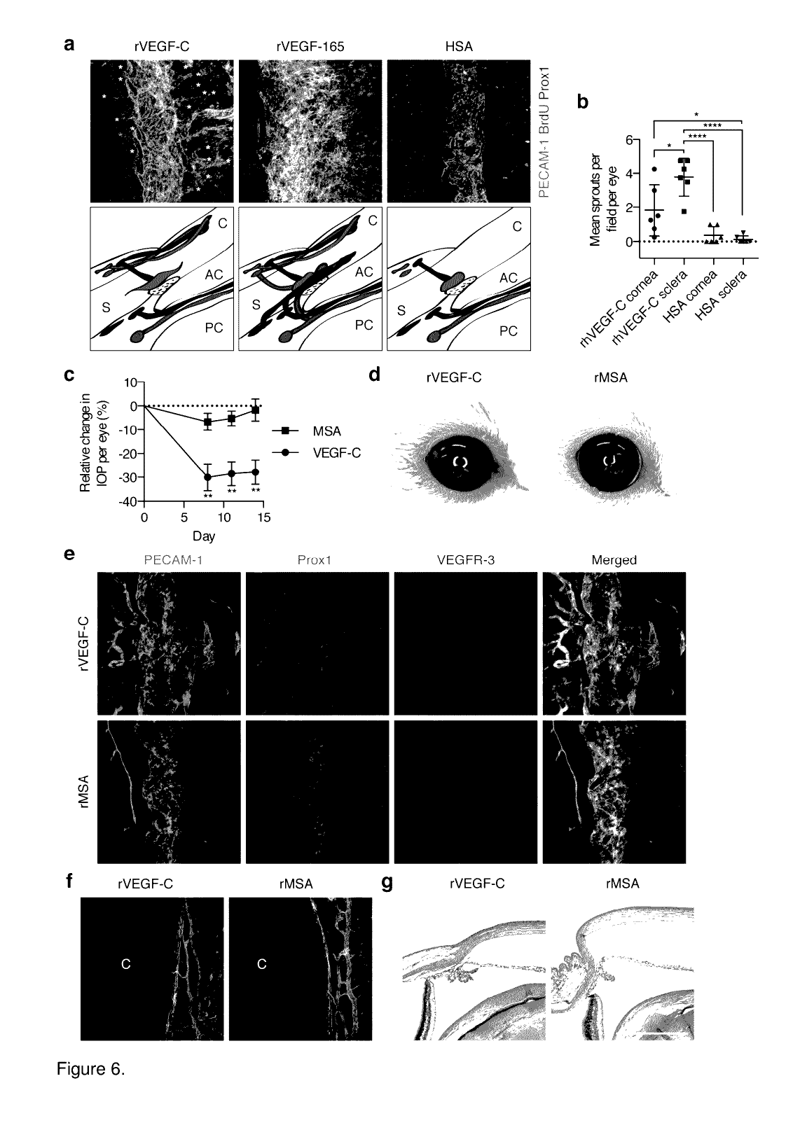

FIG. 7 shows the downregulation of Prox1 and VEGFR-3 in SC endothelium in proximity of the long posterior ciliary artery. (a) The SC and the long posterior ciliary artery (indicated by dashed line) were visualized by immunofluorescence staining using antibodies against PECAM-1, Prox1 and VEGFR-3 in adult mice. Low-magnification images, with inset 1 indicating SC area in proximity of the long posterior ciliary artery and inset 2 indicating normal SC endothelium. High-magnification images of the indicated SC area by inset 1 and inset 2. Downregulation of VEGFR-3 and Prox1 is indicated by arrowhead. (b) The SC in Prox1-mOrange mice at 18 months of age. (c) Negative control for Prox1 staining in FIG. 1f. Scale bars, 200 μm (a-c).

FIG. 8 reveals that the Schlemm's canal develops postnatally from transscleral veins and becomes blind-ended postnatally. (a) Representative images of the developing SC (indicated by red dashed line) at P0, P1, P2, P4 and P7 as visualized by immunofluorescence staining in thick sections with antibodies against PECAM-1 (green), Prox1 (red), VEGFR-2 (white), DAPI (blue) and in H&E stained sections. ES, episclera. CC, choriocapillaries. SC, Schlemm's canal. TV, transscleral vessel. S, sclera. R, retina. I, iris. * indicates Prox1 expression. (b) Representative images of the developing SC at P1, P2, P3 and P4 visualized by whole mount immunofluorescence staining with an antibody against PECAM-1 in lectin perfused pups. Arrowhead indicates lectin perfusion through transscleral vein. Note the absence of perfused transscleral veins at P4. Scale bars, 100 μm.

FIG. 9 demonstrates that Vegfc haploinsufficiency is not sufficient to inhibit SC development. (a) Immunofluoresence staining of Vegfc^{+/LacZ} and wild type littermate SC and ES lymphatics of using antibodies against PECAM-1 (green), Prox1 (red) and VEGFR-3 (blue). ES lymphatics are indicated by *. (b) VEGF-C expression (indicated by arrowheads) detected by X-gal staining in Vegfc^{+/LacZ} reporter mice. SC is indicated by dashed line. I, Iris. C, Cornea. AC, anterior chamber. ES+C, episclera and conjunctiva. Quantification analysis of the PECAM-1-positive SC area (c) and VEGFR-3 and (d) Prox1-positive ES lymphatic area in Vegfc^{+/LacZ} and wild type (WT) littermate mice. Data represent mean±s.e.m surface area in 0.225-mm² (c) and 0.900-mm² (d) limbal area (n=4 per genotype). Scale bars, 200 μm (A-P); 50 μm (Q). n.s. P>0.05, **P<0.005, two-sample (unpaired Student's) two-sided t test assuming equal variance.

FIG. 10 shows normal SC development in Chy mice. (A) Immunofluorescence staining of the murine SC and ES vasculature around the limbus in Chy (n=4) and wild type littermates (WT, n=4) mice using antibodies against PECAM-1 (green) and Prox1 (red). Quantification of PECAM-1-positive SC surface area (B) and PECAM-1 and Prox1-positive ES lymphatic surface area (C) shown in A. Data represent mean±s.e.m surface area in 0.225-mm² (B) and 0.900-mm² (C) limbal area. n.s. P>0.05, ***P<0.001, two-sample (unpaired Student's) two-sided t test assuming equal variance. Scale bars, 200 μm.

FIG. 11 demonstrates corneal neovascularization in AdVEGF-C and AdVEGF-165 injected eyes. (a-b) Immunofluorescence staining of the corneal vasculature around the limbus in AdVEGF-C, AdVEGF-165 and AdCMV injected eyes, and in uninjected eyes using antibodies against PECAM-1 (green), BrdU (red) and Prox1 (blue) at 4 days and 14 days after injection. Prior to sacrifice, the mice received on injection of 100 mg/kg of BrdU to label proliferating cells. (c) Intraocular pressure in untreated NMRI nu/nu (n=113 eyes) and NMRI (n=40) mice. ****P<0.0001, two-sample (unpaired Student's) two-sided t test assuming equal variance.

### DETAILED DESCRIPTION OF THE INVENTION

In the present invention, we establish the SC as a component of the lymphatic vascular system by demonstrating expression of lymphatic vessel markers Prox1, VEGFR-3, LYVE-1 and CCL21 by the SC endothelium in mice. We demonstrate that the development of the SC occurs in a similar, yet distinct manner to the development of the lymph sacs¹⁵. The SC morphogenesis begins when a network of limbal transcleral veins begin to sprout laterally to connect to each other and form a primordial SC. Unlike in the cardinal veins where Prox1 is induced in a subset of the venous ECs¹⁶, Prox1 is induced in the SC only after the formation of the primordial SC. This is quickly followed by subsequent upregulation of VEGFR-3. The development of the SC represents an exception to the concept that all LECs are derived from lymph sacs¹⁷. In the developing lymphatic vessels, VEGF-C is required for the migration of Prox1-expressing initial LECs^{15,16}. Analogously, we show here that conditional deletion of VEGF-C or concomitant inhibition of VEGF-C and VEGF-D by the soluble VEGF-C/D trap in K14-VEGFR-3(1-3)-Ig inhibits migration of endothelial cells committed to the SC lineage. By conditionally deleting Vegfr3 in the SC ECs, we demonstrate a critical role for VEGFR-3 in SC development. Furthermore, we show that at least the initial stages of SC development involve VEGFR-2, as it is expressed in the initial transscleral vessels and throughout SC development, and blocking VEGFR-2 with monoclonal antibodies inhibits SC growth.

Prompted by these findings, we performed experiments in adult mice to show that overexpression of VEGF-C in the anterior chamber of the eye in adult mice results in the sprouting, proliferation and migration of SC ECs. Most strikingly, we show that the administration of a single injection of recombinant VEGF-C results in a sustained decrease of IOP without inducing corneal neovascularization or other pathologies. Reductions in IOP in normotensive mice have been shown to accurately predict positive treatment responses in glaucoma^{18,19}. VEGFR-3 activating ligand results in decreasing IOP thus representing a potential curative form of treatment for glaucoma. Collectively, these results represent major conceptual advances in lymphatic vascular biology and open novel therapeutic avenues in the treatment of glaucoma.

### Ocular Hypertension and Glaucoma

As set forth above, the present therapeutic methods and uses relate to the treatment of ocular hypertension or glaucoma. As used herein, the term “treatment” or “treating” refers to administration of a VEGFR-3 ligand, i.e. at least VEGF-C, to a subject, preferably a mammal or human subject, for purposes which include not only complete cure but also prophylaxis, amelioration, or alleviation of disorders or symptoms related to ocular hypertension or glaucoma. Therapeutic effect of administration of a VEGFR-3 ligand may be assessed by monitoring symptoms such as IOP, pain or impaired vision.

Glaucoma is a term describing a group of ocular disorders with multi-factorial etiology united by a clinically characteristic intraocular pressure-associated optic neuropathy (Casson, R J et al. (2012). Clinical & Experimental Ophthalmology 40 (4): 341-9.). Glaucoma is characterized by chronic, degenerative optic neuropathy in which loss of axons and supporting structures leads to a characteristic excavation of the optic nerve head with resultant loss of visual field¹. Thus glaucoma can permanently damage vision in the affected eye(s) and lead to blindness if left untreated.

Glaucoma has been classified into specific types (Paton D and Craig J A (1976). Glaucomas. Clin Symp 28 (2): 1-47) and can be selected from the group consisting of primary glaucoma and its variants, developmental glaucoma, secondary glaucoma and absolute glaucoma. As used herein “primary glaucoma” includes primary angle closure glaucoma (such as acute angle closure glaucoma, chronic angle closure glaucoma, intermittent angle closure glaucoma or superimposed on chronic open-angle closure glaucoma) and primary open-angle glaucoma (such as high-tension glaucoma or low-tension glaucoma). As used herein “variants of primary glaucoma” include pigmentary glaucoma and exfoliation glaucoma. As used herein “developmental glaucoma” includes primary congenital glaucoma, infantile glaucoma and glaucoma associated with hereditary of familial diseases. As used herein “secondary glaucoma” includes inflammatory glaucoma (such as uveitis of all types or fuchs heterochromic iridocyclitis), phacogenic glaucoma (such as angle-closure glaucoma with mature cataract, phacoanaphylactic glaucoma secondary to rupture of lens capsule, phacolytic glaucoma due to phacotoxic meshwork blockage, subluxation of lens), glaucoma secondary to intraocular hemorrhage (such as hyphema or hemolytic glaucoma), traumatic glaucoma (such as angle recession glaucoma or postsurgical glaucoma (such as aphakic pupillary block or ciliary block glaucoma)), neovascular glaucoma, drug-induced glaucoma (such as corticosteroid induced glaucoma or alpha-chymotrypsin glaucoma) and glaucoma of miscellaneous origin (such as associated with intraocular tumors, associated with retinal detachments, secondary to severe chemical burns of the eye, associated with essential iris atrophy or toxic glaucoma). As used herein “absolute glaucoma” refers to the end stage of all types of glaucoma.

Ocular hypertension (OHT) is intraocular pressure higher than normal in the absence of optic nerve damage or visual field loss. As used herein “intraocular pressure higher than normal” refers to intraocular pressure levels above 21 mm Hg. Elevated IOP is the most important risk factor for glaucoma. Therefore those with ocular hypertension are considered to have a greater chance of developing glaucoma. Ocular hypotensive medication (e.g. topical medication) may be used in delaying or preventing the onset of POAG in individuals with elevated IOP⁸. Although this does not imply that all patients with borderline or elevated IOP should receive medication, clinicians should consider initiating treatment for individuals with ocular hypertension who are at moderate or high risk for developing POAG.

### VEGFR-3 Ligands

Vascular endothelial growth factor C (VEGF-C) is one of the main drivers of lymphangiogenesis in embryonic development and in various lymphangiogenic processes in adults (Alitalo, 2011, Nature Medicine 17: 1371-1380). VEGF-C acts by activating VEGFR-3 and—in its proteolytically processed mature forms—also VEGFR-2. Deletion of the Vegfc gene in mice results in failure of lymphatic development due to the inability of newly differentiated lymphatic endothelial cells to migrate from the central veins to sites where the first lymphatic structures form (Karkkainen et al, 2003, Nature Immunology 5: 74-80; Hägerling et al, 2013, EMBO J 32: 629-644). This phenotype could be rescued by the application of VEGF-C (Karkkainen et al, 2003, ibid.). For the rescue, a “mature” recombinant form of VEGF-C was used, which lacked the N- and C-terminal propeptides. In cells secreting endogenous VEGF-C, these propeptides need to be proteolytically cleaved off from the central VEGF homology domain (VHD) in order for VEGF-C to reach its full signaling potential (Joukov et al, 1997, EMBO J 16: 3898-3911). VEGF-C can activate the main angiogenic receptor VEGFR-2 significantly only when both propeptides are cleaved off (Joukov et al, 1997, ibid.) and hence, the mature VEGF-C stimulates also angiogenesis.

As used herein, the term “VEGFR-3 ligand” or “VEGFR-3 activating ligand” refers to any VEGF-C. VEGFR-3 ligands include but are not limited to any VEGF-C polypeptide, or VEGF-C polynucleotide including for example any variants of VEGF-C and recombinant VEGF-C. VEGFR-3 activating ligands bind VEGFR-3 and thereby increase VEGFR-3 signalling resulting in increased lymphangiogenesis or angiogenesis.

As used herein, the term “VEGF-C” refers to any VEGF-C, such as any VEGF-C polypeptide or VEGF-C polynucleotide including for example any variants of VEGF-C and recombinant VEGF-C's.

As used herein, the term “VEGF-C polypeptide” refers to any known form of VEGF-C including prepro-VEGF-C, partially processed VEGF-C, and fully processed mature VEGF-C. During its biosynthesis, the full-length form of VEGF-C (58 kDa) first undergoes a proteolytic cleavage in the C-terminal part, resulting in the 29/31 kDa intermediate form held together via disulfide bonds, and a subsequent cleavage at two alternative sites in the N-terminus, yielding the mature, fully active 21 kDa or 23 kDa form of VEGF-C. This process is known to be inefficient, as the majority of VEGF-C protein does not become activated. However, the difference in the lymphangiogenic potential between the mature and the 29/31 kDa intermediate forms is remarkable (Anisimov et al, 2009, Circulation Research 104:1302-1312).

In some embodiments, the VEGF-C polypeptide to be used therapeutically in accordance with the present invention is the full-length, or prepro, form of VEGF-C. In some further non-limiting embodiments, the prepro-VEGF-C polypeptide lacks a signal sequence and, thus, may comprise amino acids 32-419 of the sequence depicted in SEQ ID NO:2, for instance. A person skilled in the art realizes that there are alternative cleavage sites for signal peptidases and that other proteases may process the N-terminus of VEGF-C without affecting the activity thereof. Consequently, the VEGF-C polypeptide may differ from that comprising or consisting of amino acids 32-419 of SEQ ID NO: 2.

Alternatively or additionally, the VEGF-C polypeptide may be in the form of a partly processed VEGF-C, such as that comprising amino acids 32-227 covalently linked to amino acids 228-419 of the amino acid sequence depicted in SEQ ID NO: 2. Again, owing to alternative cleavage sites for signal peptidases and other proteases, the partially processed VEGF-C polypeptide may have an amino acid composition different from that of the non-limiting example described above without deviating from the present invention and its embodiments.

In some still further embodiments, the VEGF-C polypeptide to be administered to a subject suffering from ocular hypertension or glaucoma is in the fully processed, or mature, form thereof. For example VEGF-C may comprise amino acids 112-227 or 103-227 of the amino acid sequence depicted in SEQ ID NO: 2. Further, the VEGF-C polypeptide may be in any other naturally occurring or engineered form. If desired, different forms of VEGF-C polypeptides may be used in any combination. In a specific embodiment, the VEGF-C polypeptide is a mammalian VEGF-C polypeptide, e.g. an animal or human VEGF-C polypeptide.

It is also contemplated that any of the VEGF-C polypeptides described herein may vary in their amino acid sequence as long as they retain their biological activity, particularly their capability to bind and activate VEGFR2 and/or VEGFR-3. Therefore, as used herein VEGF-C polypeptide also refers to any fragment of VEGF-C polypeptide capable of binding to and activating VEGFR-2 and/or VEGFR-3. In some embodiments, the VEGF-C may be a conservative sequence variant of any VEGF-C polypeptide, respectively, described herein or it may comprise an amino acid sequence that is at least 85%, 90%, 95%, 96%, 97%, 98%, 99% or more identical to the amino acid sequence depicted in SEQ ID NO: 2 or SEQ ID NO: 4, respectively, or any biologically relevant fragment thereof.

As used herein, the term “VEGF-C polynucleotide” refers to any polynucleotide, such as single or double-stranded DNA or RNA, comprising a nucleic acid sequence encoding any VEGF-C polypeptide. As used herein VEGF-C polynucleotide also refers to any polynucleotide encoding a fragment of VEGF-C polypeptide capable of binding to and activating VEGFR-2 and/or VEGFR-3. For instance, the VEGF-C polynucleotide may encode a full-length VEGF-C and comprise or consists of nucleic acids 524-1687 of a nucleic acid sequence depicted in SEQ ID NO: 3. In some other embodiments, the VEGF-C polynucleotide may encode intermediate forms of VEGF-C and comprise or consists of either nucleic acids 737-1687 or 764-1687 of the nucleic acid sequence depicted in SEQ ID NO: 3. In some further embodiments, the VEGF-C polynucleotide may encode mature forms of VEGF-C and comprise or consists of either nucleic acids 737-1111 or 764-1111 of the nucleic acid sequence depicted in SEQ ID NO: 3. None of the above embodiments contains sequences encoding a signal peptide or a stop codon but other embodiments may comprise such sequences. In some still further embodiments, the C-terminus of the mature forms may be shortened without losing receptor activation potential.

Conservative sequence variant of said nucleic acid sequences are also contemplated. In connection with polynucleotides, the term “conservative sequence variant” refers to nucleotide sequence modifications, which do not significantly alter biological properties of the encoded polypeptide. Conservative nucleotide sequence variants include variants arising from the degeneration of the genetic code and from silent mutations.

Nucleotide substitutions, deletions and additions are also contemplated. Accordingly, multiple VEGF-C encoding polynucleotide sequences exist for any given VEGF-C polypeptide, any of which may be used therapeutically as described herein.

In some further embodiments, the VEGF-C polynucleotide may comprise a nucleic acid sequence which is at least 85%, 90%, 95%, 96%, 97%, 98%, 99% or more identical to the VEGF-C nucleic acid sequences described above, as long as it encodes a VEGF-C polypeptide that has retained its biological activity, particularly the capability to bind and activate VEGFR-2 and VEGFR-3.

Preferably, any VEGF-C polynucleotide described herein comprises an additional N-terminal nucleotide sequence motif encoding a secretory signal peptide operably linked to the polynucleotide sequence. The secretory signal peptide, typically comprised of a chain of approximately 5 to 30 amino acids, directs the transport of the polypeptide outside the cell through the endoplasmic reticulum, and is cleaved from the secreted polypeptide. Suitable signal peptide sequences include those native for VEGF-C, those derived from another secreted proteins, such as CD33, Ig kappa, or IL-3, and synthetic signal sequences.

A VEGF-C polynucleotide may also comprise a suitable promoter and/or enhancer sequence for expression in the target cells, said sequence being operatively linked upstream of the coding sequence. If desired, the promoter may be an inducible promoter or a cell type specific promoter, such as an endothelial cell specific promoter. Suitable promoter and/or enhancer sequences are readily available in the art and include, but are not limited to, EF1, CMV, and CAG.

Furthermore, any VEGF-C polynucleotide described herein may comprise a suitable polyadenylation sequence operably linked downstream of the coding sequence.

VEGF-C of the present invention may be an animal, mammal or human VEGF-C. In a specific embodiment of the invention, VEGF-C is a human VEGF-C.

In one embodiment of the invention the VEGFR-3 activating ligand is in a form of a fusion protein. VEGF-C may be delivered to a subject as a fusion protein of VEGF-C and any other protein. For example VEGF-C/angiopoietin 1 or VEGF-C/angiopoietin 2 fusion proteins, VEGF/VEGF-C mosaic molecules (described in J Biol Chem. 2006 Apr. 28; 281(17):12187-95), chimeric VEGF-C/VEGF heparin-binding domain fusion proteins (described in Circ Res. 2007 May 25; 100(10):1468-75), chimeric VEGF/VEGF-C silk domain fusion proteins (described in Circ Res. 2007 May 25; 100(10):1460-7) and VEGF-angiopoietin chimeras (described in Circulation 2013 Jan. 29; 127(4):424-34) can be utilized for the present invention.

### Administration

Therapeutic use of VEGFR-3 ligands may be implemented in various ways, for instance by gene therapy, protein therapy, or any desired combination thereof. Administration of VEGFR-3 ligands by different ways or routes may be simultaneous, separate, or sequential.

VEGF-C may be the only therapeutically effective agent (i.e. having an ability to ameliorate any harmful effects of ocular hypertension or glaucoma) used for treatments of the present invention. In one embodiment of the invention VEGF-C is the only therapeutically effective agent(s). VEGF-C may also be administered together with other agents, such as therapeutically effective agents. In one embodiment of the invention, the composition further comprises other therapeutically effective agents. For co-administration of VEGFR-3 ligand and any other agent the route and method of administration may be selected independently. Further, co-administration of VEGFR-3 ligand and any other therapeutically effective agent may be simultaneous, separate, or sequential. In one embodiment of the invention, the VEGFR3 activating ligand or the composition is used concurrently with other therapeutic agents or therapeutic methods, such as a surgical method.

As used herein, the term “gene therapy” refers to the transfer of a VEGF-C polynucleotide into selected target cells or tissues in a manner that enables expression thereof in a therapeutically effective amount. In accordance with the present invention, gene therapy may be used to replace a defective gene, or supplement a gene product that is not produced in a therapeutically effective amount or at a therapeutically useful time in a subject with ocular hypertension or glaucoma.

As used herein, the term “subject” refers to a subject, which is selected from a group consisting of an animal, a mammal or a human. In one embodiment of the invention, the subject is a human or an animal. Before classifying a human or animal patient as suitable for the therapy of the present invention, for example elevated IOP may be assayed or the level of pain or impaired vision may be studied. After these preliminary studies and based on the results deviating from the normal, the clinician may suggest VEGFR3 ligand treatment for a patient. Patients may be selected for the treatments or therapies of the present invention for example based on any detectable or noticeable disorder such as increased IOP, pain or impaired vision.

As used herein, the term “protein therapy” refers to the administration of a VEGF-C polypeptide in a therapeutically effective amount to a subject, particularly a mammal or a human, with ocular hypertension or glaucoma for which therapy is sought. Herein, the terms “polypeptide” and “protein” are used interchangeably to refer to polymers of amino acids of any length.

As used herein, the term “therapeutically effective amount” refers to an amount of VEGF-C with which the harmful effects of ocular hypertension or glaucoma are, at a minimum, ameliorated. The harmful effects of ocular hypertension or glaucoma include any detectable or noticeable effects of a subject such as increased IOP, pain or impaired vision.

For gene therapy, “naked” VEGF-C polynucleotides described above may be applied in the form of recombinant DNA, plasmids, or viral vectors. Delivery of naked polynucleotides may be performed by any method that physically or chemically permeabilizes the cell membrane. Such methods are available in the art and include, but are not limited to, electroporation, gene bombardment, sonoporation, magnetofection, lipofection, liposome-mediated nucleic acid delivery, and any combination thereof.

In some other embodiments, VEGF-C polynucleotides may be incorporated into a viral vector under a suitable expression control sequence. Suitable viral vectors for such gene therapy include, but are not limited to, retroviral vectors, such as lentivirus vectors, adeno-associated viral vectors, and adenoviral vectors. Preferably, the viral vector is a replication-deficient viral vector, i.e. a vector that cannot replicate in a mammalian subject. A non-limiting preferred example of such a replication-deficient vector is a replication-deficient adenovirus. Suitable viral vectors are readily available in the art. In the specific embodiment of the invention, the VEGF-C is overexpressed by adenoviral or adeno-associated viral vectors.

Delivery of therapeutic VEGF-C polynucleotides to a subject, preferably a mammalian or a human subject, may be accomplished by various ways well known in the art. For instance, viral vectors comprising VEGF-C encoding polynucleotide(s) may be administered directly into the body of the subject to be treated, e.g. by an injection into an eye (e.g. anterior chamber), SC or a target tissue having compromised lymphatic vessels or into the surgically generated outflow tract. In one embodiment the target cells are endothelial cells of the SC or the target cell environment is environment of endothelial cells of the SC.

Such delivery results in the expression of the polypeptides in vivo and is, thus, often referred to as in vivo gene therapy. Alternatively or additionally, delivery of the present therapeutic polypeptides may be effected ex vivo by use of viral vectors or naked polypeptides. Ex vivo gene therapy means that target cells, preferably obtained from the subject to be treated, are transfected (or transduced with viruses) with the present polynucleotides ex vivo and then administered to the subject for therapeutic purposes. Non-limiting examples of suitable target cells for ex vivo gene therapy include endothelial cells, endothelial progenitor cells, smooth muscle cells, leukocytes, and especially stem cells of various kinds.

In gene therapy, expression of VEGF-C may be either stable or transient. Transient expression is often preferred. A person skilled in the art knows when and how to employ either stable or transient gene therapy.

In addition to gene therapy, also protein therapy aims at the sprouting and proliferation of the SC endothelial cells. For protein therapy, VEGF-C may be obtained for example by standard recombinant methods. A desired polynucleotide may be cloned into a suitable expression vector and expressed in a compatible host according to methods well known in the art. Examples of suitable hosts include but are not limited to bacteria (such as E. coli), yeast (such as S. cerevisiae), insect cells (such as SF9 cells), and preferably mammalian cell lines. Expression tags, such as His-tags, hemagglutinin epitopes (HA-tags) or glutathione-S-transferase epitopes (GST-tags), may be used to facilitate the purification of VEGF-C. If expression tags are to be utilized, they have to be cleaved off prior to administration to a subject in need thereof.

In one embodiment of the invention VEGF-C protein is administered directly to the target tissue (e.g. compromised lymphatic vessels or SC), into the anterior chamber or to the surgically generated outflow tract.

Amounts and regimens for therapeutic administration of VEGF-C according to the present invention can be determined readily by those skilled in the clinical art of treating ocular hypertension or glaucoma. Generally, the dosage of the VEGF-C treatment will vary depending on considerations such as: age, gender and general health of the patient to be treated; kind of concurrent treatment, if any; frequency of treatment and nature of the effect desired; extent of tissue damage or glaucoma or hypertension; type of glaucoma; duration of the symptoms; and other variables to be adjusted by the individual physician. For instance, when viral vectors are to be used for gene delivery, the vector is typically administered, optionally in a pharmaceutically acceptable carrier, in an amount of 10⁷ to 10¹³ viral particles, preferably in an amount of at least 10⁹ viral particles. On the other hand, when protein therapy is to be employed, a typical dose is in the range of 0.01 to 20 mg/kg, more preferably in the range of 0.1 to 10 mg/kg, most preferably 0.5 to 5 mg/kg.

A desired dosage can be administered in one or more doses at suitable intervals to obtain the desired results. A typical non-limiting daily dose may vary from about 50 mg/day to about 300 mg/day. Indeed, only one administration of VEGF-C may have therapeutic effects. However, in one embodiment of the invention, VEGF-C is administered several times during the treatment period. VEGF-C may be administered for example from 1 to 20 times, 1 to 10 times or two to eight times in the first 2 weeks, 4 weeks, monthly or during the treatment period. The length of the treatment period may vary, and may, for example, last from a single administration to 1-12 months or more.

### Pharmaceutical Compositions

The present invention provides not only therapeutic methods and uses for treating disorders and conditions related to impaired lymphatic vasculature but also to pharmaceutical compositions for use in said methods and therapeutic uses. Such pharmaceutical compositions comprise VEGF-C, either alone or in combination with other agents such as a therapeutically effective agent or agents and/or a pharmaceutically acceptable vehicle or vehicles. A pharmaceutically acceptable vehicle may for example be selected from the group consisting of a pharmaceutically acceptable solvent, diluent, adjuvant, excipient, buffer, carrier, antiseptic, filling, stabilising agent and thickening agent. Optionally, any other components normally found in corresponding products may be included. In one embodiment of the invention the pharmaceutical composition comprises VEGF-C and a pharmaceutically acceptable vehicle.

For instance, the pharmaceutically acceptable vehicle may be a sterile non-aqueous carrier such as propylene glycol, polyethylene glycol, or injectable organic ester. Suitable aqueous carriers include, but are not limited to, water, saline, phosphate buffered saline, and Ringer's dextrose solution.

A variety of administration routes may be used to achieve an effective dosage to the desired site of action as well known in the art. Thus, suitable routes of administration include, but are not limited to, subconjunctival delivery, local administration (e.g. to the eye or surgical site) and/or topical administration (e.g. on the eye), as known to a person skilled in the art.

The pharmaceutical composition may be provided in a concentrated form or in a form of a powder to be reconstituted on demand. Furthermore, the pharmaceutical composition may be in any form, such as solid, semisolid or liquid form, suitable for administration. A formulation can be selected from a group consisting of, but not limited to, for example solutions, emulsions, suspensions, tablets, pellets and capsules. A formulation may also be any matrix formulation or for example biodegradable material such as a bioimplant. The formulation may release VEGFR-3 ligand to the tissue either quickly or slowly. In case of lyophilizing, certain cryoprotectants are preferred, including polymers (povidones, polyethylene glycol, dextran), sugars (sucrose, glucose, lactose), amino acids (glycine, arginine, glutamic acid) and albumin. If solution for reconstitution is added to the packaging, it may consist e.g. of sterile water, sodium chloride solution, or dextrose or glucose solutions.

Means and methods for formulating the present pharmaceutical preparations are known to persons skilled in the art, and may be manufactured in a manner which is in itself known, for example, by means of conventional mixing, granulating, dissolving, lyophilizing or similar processes.

### Optional Therapeutically Effective Agents

VEGF-C may be administered to a subject in combination with other therapeutically effective agents. In addition to VEGF-C, a pharmaceutical composition of the invention may comprise at least one, two, three, four or five other therapeutically effective agents. In one embodiment of the invention, the composition further comprises CCBE1.

As used herein, the term “CCBE1” refers to a full-length collagen- and calcium-binding EGF domains 1 (CCBE1) polypeptide or to a polynucleotide encoding said full-length CCBE1. In one embodiment, CCBE1 is a mammalian or human CCBE1. In some embodiments, the full-length CCBE1 polypeptide does not have a signal peptide. When CCBE1 is produced in mammalian cells, the signal peptide is automatically cleaved off correctly.

It is evident to a person skilled in the art that the CCBE1 polypeptide to be used in accordance with the present invention may vary as long as it retains its biological activity. An exemplary way of determining whether or not a CCBE1 variant has maintained its biological activity is to determine its ability to promote cleavage of full-length VEGF-C. This may be performed e.g. by incubating cells expressing full-length VEGF-C with the CCBE1 variant in question and concluding that the CCBE1 variant has retained its biological activity if VEGF-C cleavage is enhanced. Said VEGF-C cleavage may be determined e.g. by metabolic labelling and protein-specific precipitation, such as immunoprecipitation, according to methods well known in the art. If desired, CCBE1 having an amino acid sequence depicted in SEQ ID NO: 1 may be used as a positive control.

In connection with polypeptides, the variants refers to amino acid sequence modifications, which arise from amino acid substitutions with similar amino acids well known in the art (e.g. amino acids of similar size and with similar charge properties) and which do not significantly alter the biological properties of the polypeptide in question. Amino acid deletions and additions are also contemplated.

As used herein, the term “CCBE1 polynucleotide” refers to any polynucleotide, such as single or double-stranded DNA or RNA, comprising a nucleic acid sequence encoding a CCBE1 polypeptide. In some preferred embodiments, the CCBE1 polynucleotide comprises a coding sequence (CDS) for full-length CCBE1, or a conservative sequence variant thereof.

In one embodiment of the invention, the composition further comprises VEGF-D. As used herein, the term “VEGF-D” refers to any VEGF-D, such as any VEGF-D polypeptide or VEGF-D polynucleotide including for example any variants of VEGF-D and recombinant VEGF-D's.

As used herein, the term “VEGF-D polypeptide” refers to any known form of VEGF-D including prepro-VEGF-D, partially processed VEGF-D, and fully processed mature VEGF-D.

In some embodiments, the VEGF-D polypeptide is the full-length, or prepro, form of VEGF-D. In some further non-limiting embodiments, the prepro-VEGF-D polypeptide lacks a signal sequence. A person skilled in the art realizes that there are alternative cleavage sites for signal peptidases and that other proteases may process the N-terminus of VEGF-D without affecting the activity thereof.

Alternatively or additionally, the VEGF-D polypeptide may be in the form of a partly processed VEGF-D. Again, owing to alternative cleavage sites for signal peptidases and other proteases, the partially processed VEGF-D polypeptide may have an amino acid composition different from that of the non-limiting example described above without deviating from the present invention and its embodiments.

In one preferred embodiment of the invention, the VEGF-D polypeptide comprises the amino acid sequence depicted in SEQ ID NO: 4, or any fragment thereof.

In some still further embodiments, the VEGF-D is in the fully processed, or mature, form thereof. In a specific embodiment, the VEGF-D polypeptide is a mammalian VEGF-D polypeptide, e.g. an animal or human VEGF-D polypeptide.

It is also contemplated that any of the VEGF-D polypeptides described herein may vary in their amino acid sequence as long as they retain their biological activity, particularly their capability to bind and activate VEGFR2 and/or VEGFR-3. Therefore, as used herein VEGF-D polypeptide also refers to any fragment of VEGF-D polypeptide capable of binding to and activating VEGFR-2 and/or VEGFR-3. In some embodiments, the VEGF-D may be a conservative sequence variant of any VEGF-D polypeptide, respectively, described herein or it may comprise an amino acid sequence that is at least 85%, 90%, 95%, 96%, 97%, 98%, 99% or more identical to the amino acid sequence depicted in SEQ ID NO: 4, or any biologically relevant fragment thereof.

As used herein, the term “VEGF-D polynucleotide” refers to any polynucleotide, such as single or double-stranded DNA or RNA, comprising a nucleic acid sequence encoding any VEGF-D polypeptide. As used herein VEGF-D polynucleotide also refers to any polynucleotide encoding a fragment of VEGF-D polypeptide capable of binding to and activating VEGFR-2 and/or VEGFR-3.

Conservative sequence variant of said nucleic acid sequences are also contemplated. In connection with polynucleotides, the term “conservative sequence variant” refers to nucleotide sequence modifications, which do not significantly alter biological properties of the encoded polypeptide. Conservative nucleotide sequence variants include variants arising from the degeneration of the genetic code and from silent mutations.

Nucleotide substitutions, deletions and additions are also contemplated. Accordingly, multiple VEGF-D encoding polynucleotide sequences exist for any given VEGF-D polypeptide, any of which may be used therapeutically as described herein.

In some further embodiments, the VEGF-D polynucleotide may comprise a nucleic acid sequence which is at least 85%, 90%, 95%, 96%, 97%, 98%, 99% or more identical to the VEGF-D nucleic acid sequences described above, as long as it encodes a VEGF-D polypeptide that has retained its biological activity, particularly the capability to bind and activate VEGFR-2 and VEGFR-3.

Preferably, any VEGF-D polynucleotide described herein comprises an additional N-terminal nucleotide sequence motif encoding a secretory signal peptide operably linked to the polynucleotide sequence. The secretory signal peptide, typically comprised of a chain of approximately 5 to 30 amino acids, directs the transport of the polypeptide outside the cell through the endoplasmic reticulum, and is cleaved from the secreted polypeptide. Suitable signal peptide sequences include those native for VEGF-D, those derived from another secreted proteins, such as CD33, Ig kappa, or IL-3, and synthetic signal sequences.

In addition to CCBE1 and/or VEGF-D other possible therapeutically effective agents to be used together with VEGF-C may include for example angiopoietin 1.

Any of the embodiments and features described above may apply independently to VEGF-C and optional other agents (such as CCBE1 and/or VEGF-D) and may be used in any desired combination. Thus, at least VEGF-C may be delivered by gene therapy or protein therapy. It is also contemplated that VEGF-C may be administered using both gene therapy and protein therapy.

It will be obvious to a person skilled in the art that, as technology advances, the inventive concept can be implemented in various ways. The invention and its embodiments are not limited to the examples described below but may vary within the scope of the claims.

### Examples

### Materials and Methods

### Antibodies

The following primary antibodies were used for immunostaining of mouse tissues: rabbit anti-mouse Prox1²⁰ (1:200), goat anti-human Prox1 (R&D, AF2727, 1:500) polyclonal goat anti-mouse VEGFR-3 (AF743, R&D Systems, 1:50), unconjugated rat anti-PECAM-1 (clone MEC 13.3, 553370, BD Pharmingen, 1:500), hamster anti-PECAM-1 (clone 2H8, MAB1398Z, Chemicon, 1:500), Cy3-conjugated mouse anti-SMA (clone 1A4, C6189, Sigma), polyclonal rabbit anti-LYVE-1¹⁶, goat anti-CCL21 (AF457, R&D Systems, 1:100), and VE-Cadherin (clone 11 D4.1, BD Pharminogen, 1:100). The primary antibodies were detected with the appropriate Alexa 488, 594 or 647 secondary antibody conjugates (Molecular Probes/Invitrogen). Bromodeoxyuridine (BrdU) was detected with Alexa 594-conjugated mouse anti-BrdU antibodies (Molecular Probes/Invitrogen) after incubation in hydrochloric acid and neutralization using sodium tetraborate. For staining in human sections, biotinylated rabbit anti-goat IgG (BA-5000, Vector Laboratories, 1:300) antibody was used.

### Mice and Tissues

All animal experiments were approved by the Committee for Animal Experiments of the District of Southern Finland and conformed to the Association for Research in Vision and Ophthalmology Statement for the Use of Animals in Ophthalmic and Vision Research. The Vegfc^{+/LacZ 16,} K14-VEGFR-3(1-3)-Ig²¹ and K14-VEGFR-3(4-7)-Ig²² VEGF-D^{-/-} (Ref. ²³), Chy²⁴, Vegfr3^{flox/flox} (Ref. ²⁵), Vegfr2^{flox/flox} (Ref. ²⁶) Rosa26-CreER^{T2} (Ref. ²⁷), Prox1-CreER^{T2} (Ref. ²⁸), Prox1-mOrange²⁹, R26-flox-STOP-flox-tdTomato³⁰ mouse lines have been published previously and Vegfc^{flox/flox} mice will be reported elsewhere (Nurmi et. al., manuscript in preparation). Neonatal wild-type mice in the NMRI and NMRI nu/nu were used for the experiments. Genetic strains were in C57BL/6J background with the exception of Vegfc^{+/LacZ} mice in the ICR, Chy and VEGF-D^{-/-} mice in the NMRI and Vegfc^{flox/flox} mice were in a mixed (C57BL/6J) background. For the induction of Cre-mediated recombination in neonatal Vegfr3^{flox/flox}; Prox1-CreER^{T2}, Vegfc^{flox/flox}; R26-CreER^{T2} or control mice, 4-hydroxytamoxifen (4-OHT; 2 μl 20 mg/ml dissolved in 97% ethanol) was injected intragastrically using a 10 μl Hamilton syringe. Daily injections were performed from P0 or P1 to P6 and the vessels were analyzed at P7. Deletion efficacy was validated either by staining (Vegfr3^{flox/flox} and Vegfr2^{flox/flox}) or by RT-qPCR (Vegfc^{flox/flox}). After sacrificing the mice, tissues were immersed in 4% paraformaldehyde, washed in phosphate buffered saline (PBS) and then processed for whole-mount staining or immersed in OCT medium (Tissue Tek).

### Human Samples

The Department of Ophthalmology archives of the University of Helsinki Central Hospital were browsed for enucleated paraffin embedded eyes removed due to ocular melanoma. Two normotensive eyes without anterior chamber involvement were selected for analysis.

### Generation and In Vitro Analysis of Viral Vectors and Production of Recombinant Proteins

The adenoviruses encoding VEGF-C, VEGF165, CMV and LacZ, the adeno-associated virus (AAV) constructs encoding VEGF-C, VEGF165, HSA and GFP and the recombinant human VEGF-C and VEGF165 proteins were produced and analyzed as described previously^{16,31-34}.

### Intraocular Pressure Measurement

IOP was measured with an induction/impact tonometer (Icare® TONOLAB, Icare Finland)³⁵ that was mounted to a stand and clamp according to the manufacturers recommendations. After the mice were anesthetized with intraperitoneally administered ketamine (60 mg/kg, Ketaminol Vet, Intervet International B.V., Netherlands) and xylazine (6 mg/kg, Rompun® Vet, KVP Pharma+Veterinár Produkte GmbH, Germany), they were placed on an adjustable height platform. The platform was adjusted for each eye to be measured in order to allow the apex of the central cornea to be normal to and 2-3 mm away from the probe tip. The mean of six consecutive IOP measurements was read from the digital readout of the tonometer and repeated three times for each eye. Repeat IOP measurements were performed on the same time of the day as baseline measurements in order to avoid circadian fluctuations in the readings.

### Intraocular Injection

After baseline IOP measurements, intraocular injection of indicated preparations was performed with a 30G ½″ needle (BD Microlance™ 3, BD Drogheda, Ireland) attached to a 10 μl Hamilton microliter syringe (Model 701 LT SYR, Hamilton Company). The needle was inserted into the posterior chamber 1 mm posterior from the limbus and into the 10.30 clock position in order any blood vessels. For the recombinant proteins, 4.8 μg of protein was injected. For adenoviruses, 5.80E+07 p.f.u. was injected. For AAVs, 3,38E+09 viral particles were injected.

### Immunostaining, X-Gal Staining, BrdU Staining and Microscopy.

For whole-mount staining, the fixed anterior segment of the eye was separated in a coronal plane. The retina and lens were removed. The tissues were permeabilized in 0.3% Triton X-100 in PBS (PBS-TX), and blocked in 5% donkey serum. Primary antibodies were added to the blocking buffer and incubated with the tissue overnight at room temperature (RT). After washes in PBS-TX, the tissue was incubated with fluorophore-conjugated secondary antibodies in PBS-TX overnight at RT, followed by washing in PBS-TX. After post-fixation in 1% PFA, the tissues were washed with PBS, cut into four quadrants, and mounted. For thick cryosections, 50 μm sections of eyes were air-dried, encircled with a pap-pen and fixed in 4% PFA for 8 minutes, rehydrated in PBS and blocked with 3% BSA in PBS-TX at RT. After primary antibody incubation in +4° C. in 3% BSA in PBS overnight, sections were washed with PBS and incubated for 2-3 hours with the appropriate fluorophore-conjugated secondary antibody conjugates in 1:300 dilution in 3% BSA in PBS. After washes with 0.1% PBS-TX, sections were mounted. All fluorescently labeled samples were mounted with Vectashield mounting medium containing 4,6-diamidino-2-phenylindole (DAPI; H-1200, Vector Laboratories). For the visualization of VEGF-C expression in Vegfc^{+/LacZ} reporter mice, the tissues were fixed with 0.2% glutaraldehyde and stained by the beta-galactosidase substrate X-Gal (Promega). For BrdU stainings, mice were given 100 mg/kg of 5-bromo-2-deoxyuridine (BrdU) by intraperitoneal injections 2 h before sacrifice. For the TSA-IHC staining of human paraffin embedded eyes, section were first deparaffinated in a decreasing alcohol series (xylene, absolute ethanol, 95%, 70%, 50%, H₂O) and subjected to antigen retrieval with incubation in high pH buffer (10 mM Tris, 1 mM EDTA, 0.05% Tween-20, pH 9.0) in the microwave for 15 minutes. After washes in PBS, endogenous peroxidase activity was quenched with incubation in 3% H₂O₂-MetOH (225 ml MetOH, 25 ml H₂O₂). After washes in H₂O, the slides were mounted onto racks with PBS, blocked with TNB for 30 minutes and primary antibodies were incubated in TNB overnight in +4 C. On the second day, after washes with TNT, the appropriate biotinylated secondary antibody in TNB was incubated for 30 minutes. After washes with TNT, Streptavidin-HRP (NEL700001KT, TSA kit, Perkin Elmer) was applied for 30 minutes. After washes, Biotin Tyramide Working Solution (NEL700001KT, TSA kit, Perkin Elmer) was applied for 10 min. at RT. After washes with TNT, Streptavidin-HRP (NEL700001KT, TSA kit, Perkin Elmer) was incubated for 30 minutes. After washed in TNT, slides were taken out of racks and treated with AEC (235 ml NaAc+15 ml AEC+250 μl H₂O₂) for 10 min. After washes with PBS and rinsing with H₂O, counterstaining with hematoxylin was applied and the slides were rinsed with running water and mounted with Aqua-Mount (Thermo Scientific).

### Microscopy

Fluorescently labeled samples were analyzed with a confocal microscope (Zeiss LSM 510 Meta, objectives ×10 with NA 0.45 and oil objectives ×40 with NA 1.3; Zeiss LSM 5 Duo, objectives 10× with NA 0.45 and oil objective ×40 with NA 1.3, and Zeiss LSM 780, objectives 10× with NA 0.45, 20× with NA 0.80, oil objective 40× with NA 1.3) using multichannel scanning in frame mode, as before³⁶. The pinhole diameter was set at 1 Airy unit for detection of the Alexa 488 signal, and was adjusted for identical optical slice thickness for the fluorophores emitting at higher wavelengths. The Zeiss ZEN 2010 or the LSM AIM (Rel. 4.2) softwares were used for image acquisition. Three-dimensional projections were digitally reconstructed from confocal z stacks. Three-dimentional volume renderings and videos were generated with the Imaris software (Bitplane). Bright-field microscopy was performed with a Leica DM LB microscope (objectives ×10 with NA 0.25 and ×20 with NA 0.4) with an Olympus DP50 color camera. Images were edited using Image J or Adobe Photoshop software.

### Vessel Morphometry and Quantitative Analysis

The vascular surface areas of the SC were quantified as PECAM-1-positive area from confocal micrographs acquired of all intact quarters of the anterior segment using Image J software. For statistical analysis, the surface areas from all quadrants were averaged from one or both eyes.

### Statistical Analysis.

Quantitative data were compared between different groups by two-sample (unpaired Student's) two-tailed t test assuming equal variance or one-way ANOVA followed by Tukey post-hoc test for multiple comparisons. The values are expressed as mean±SD. Differences were considered statistically significant at P less than 0.05.

### Results

The Schlemm's Canal Lining has Molecular Characteristics of Lymphatic Endothelia.

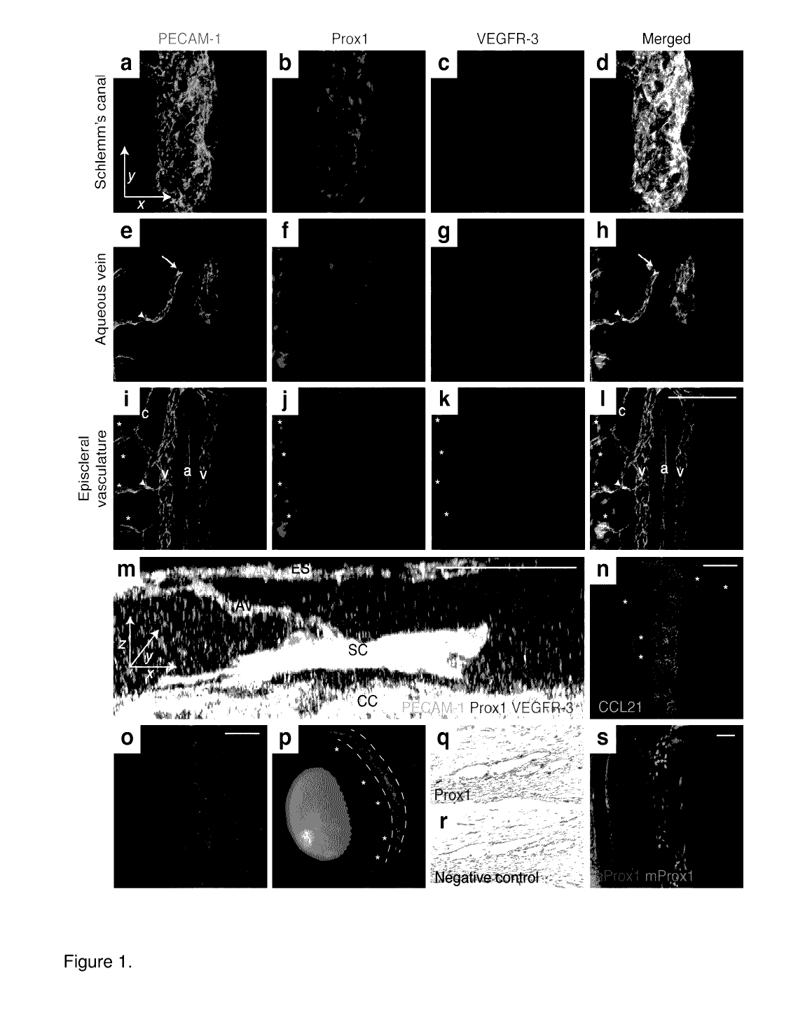

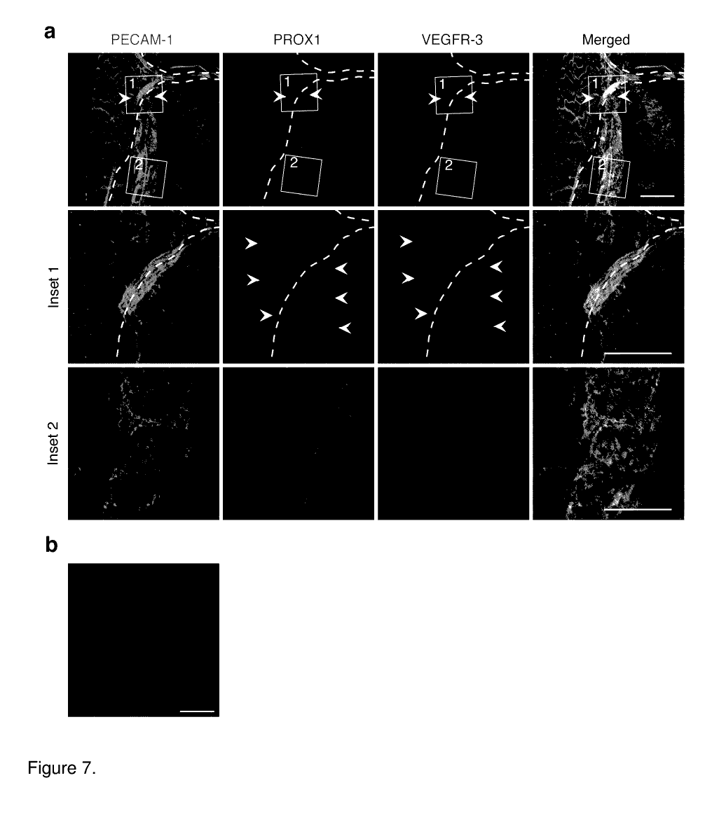

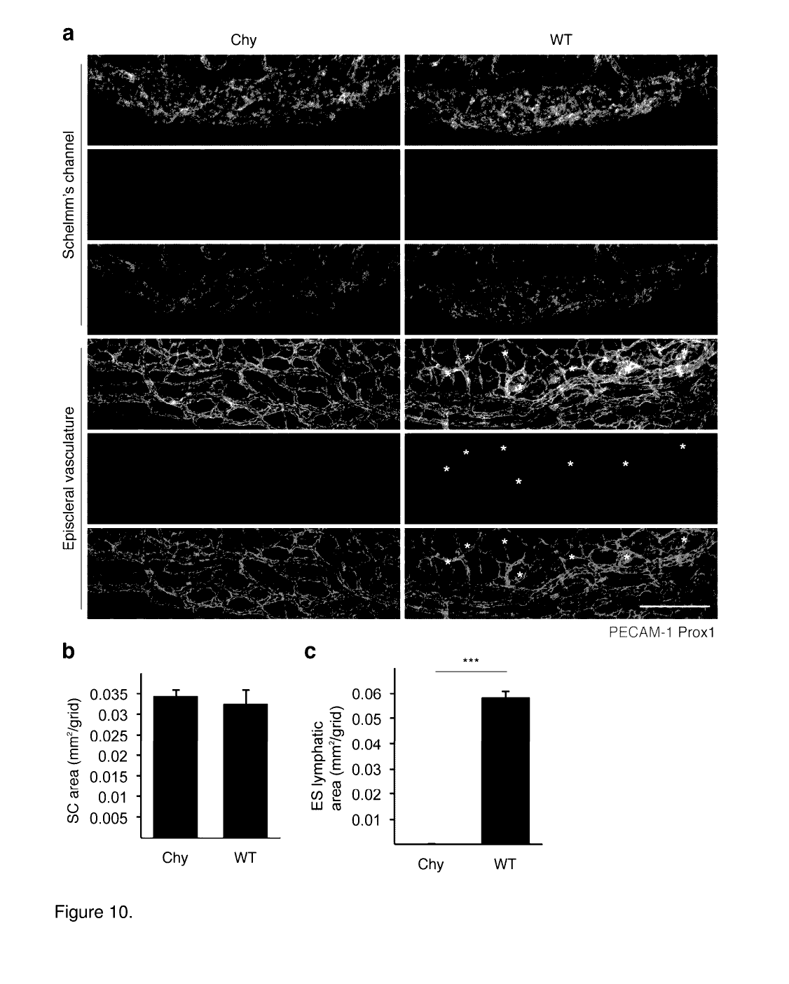

To investigate if the SC is a lymphatic vessel, we analyzed the expression of LEO markers in mouse, zebrafish and human eyes. The SC in mouse eyes was visualized using whole mount immunofluorescence staining of the eye anterior to the corneal limbus. In laser-scanning confocal microscopy (LSCM), the SC at the limbus expressed the platelet-endothelial cell adhesion molecule-1 (PECAM-1) (FIG. 1a), the lymphatic master transcription factor Prox1 (FIG. 1b) and the lymphangiogenic receptor tyrosine kinase VEGFR-3 (FIG. 1c-d). At regular intervals, the SC was observed to connect into aqueous veins (AVs) that were positive for PECAM-1 (FIG. 1e), but negative for Prox1 and VEGFR-3 (FIG. 1f-h, joining point with SC indicated by arrow). AVs were observed to drain into episcleral (ES) veins on the surface of the eye (FIG. 1i-l, joining point to ES vein indicated by arrowhead). The ES lymphatic vessels were positive and blood vessels negative for Prox1 and VEGFR-3, providing internal negative and positive controls for the stainings (FIG. 1i-l, lymphatic capillary indicated by *, artery by a, and vein by v, and capillary by c). Overall, the SC and AVs were detected between choriocapillaries (CC) and the ES vasculature (FIG. 1m), where the SC forms a uniform duct that runs at the base of the iris throughout the limbal circumference. Immunofluorescence analysis revealed strong and specific staining for the secreted chemokine ligand CCL21 (FIG. 1n) and weak expression of the lymphatic hyaluronan receptor LYVE-1 (FIG. 10) in the SC endothelium. Additionally, we detected strong Prox1 expression in the SC and ES lymphatics in up to 18 month old Prox reporter mice²⁹, which express the fluorescent protein mOrange under the Prox1 promoter (FIG. 7b). Furthermore, we mated Prox1-CreER^{T2} (Ref. ²⁸) mice with R26-loxP-STOP-loxP-tdTomato³⁰ Cre reporter mice to visualize the SC in vivo and to validate that the Prox1-CreER^{T2} allele could be used to achieve SC-specific tamoxifen-inducible conditional gene deletion in the SC endothelium. In these mice, the SC and episcleral lymphatic vessels were specifically labeled (FIG. 1p, SC indicated by dashed line, episcleral lymphatic vessel by *).

Strong Prox1 expression was detected also in human SC endothelium (FIG. 1q-r). Furthermore, the zebrafish SC could be visualized using whole mount immunofluorescence staining with two independent Prox1 antibodies (FIG. 1s), indicating that the lymphatic identity of the SC is conserved in vertebrate evolution.

The Schlemm's Canal Develops Postnatally from Transscleral Veins.

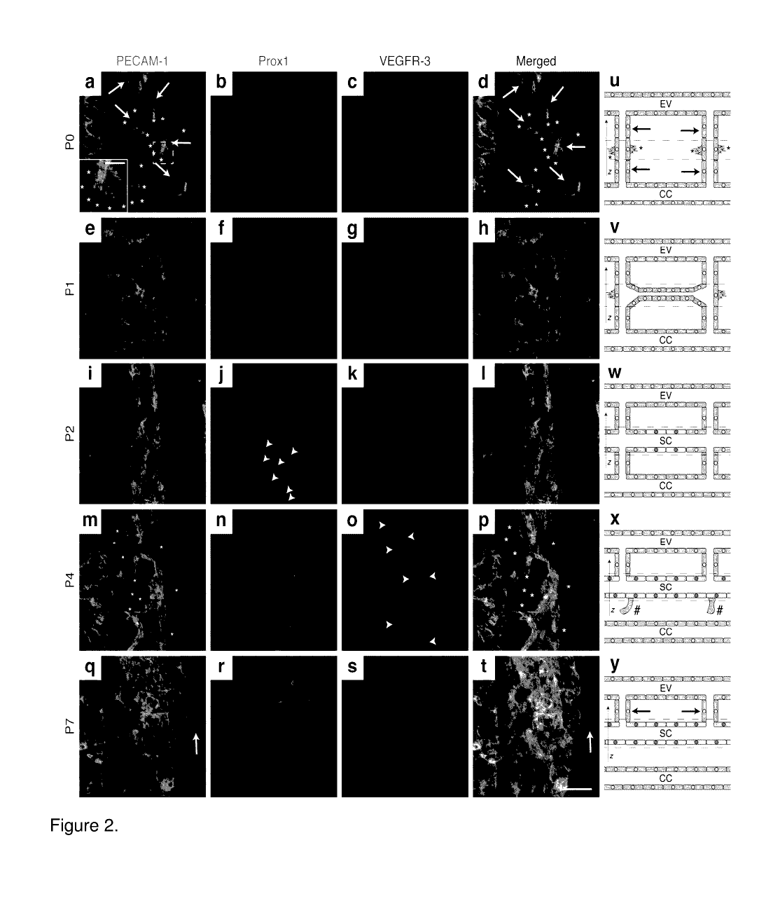

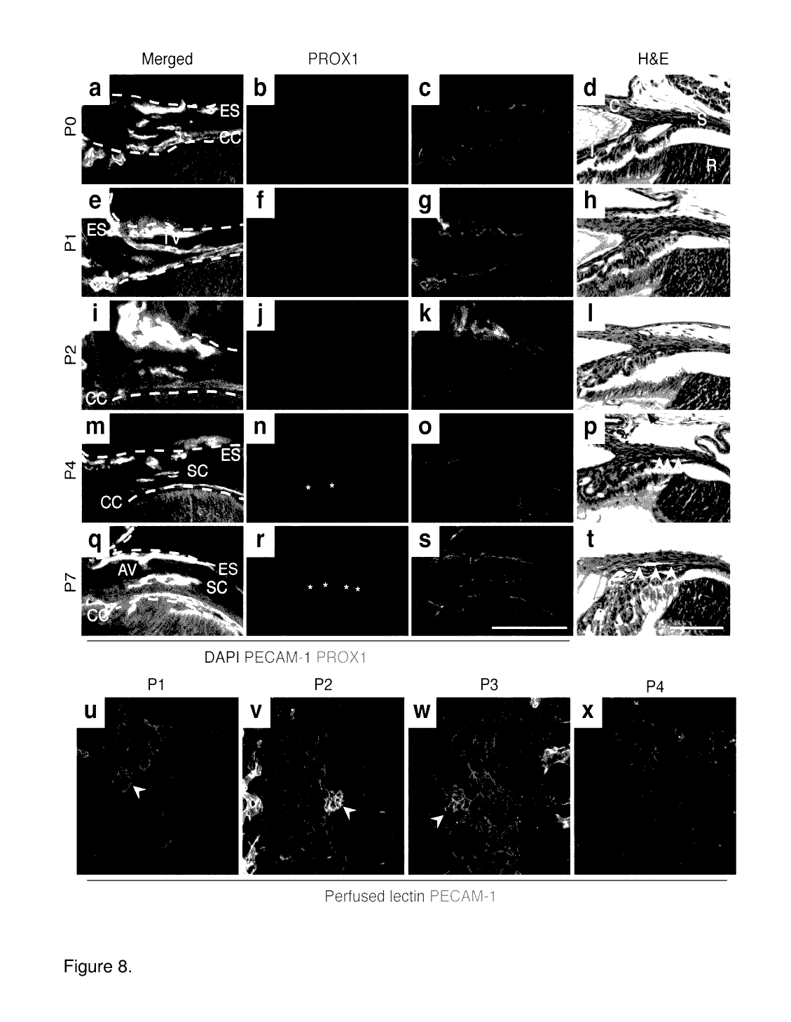

The characterization of the SC developmental morphogenesis has previously been limited to serial sections³⁷, which do not provide enough information. The development of the lymph sacs has recently been re-characterized by applying selective plane illumination-based ultramicroscopy¹⁵. We next set out to visualize the development of the SC in mice by applying LSCM to whole mount immunofluorescence stained samples (FIG. 2), which allowed us to generate 3D volume renderings of the confocal stacks. The formation of the SC was traced back to postnatal (P) day 0, when a circular network of limbal CC sprouts toward ES veins and transscleral vessels connecting CCs and ES veins was observed (FIG. 2a-e, u, Supplementary Video 1, FIG. 8a-d). At P1, these transscleral vessels had begun to sprout toward each other to form a disorganized network at the site of the future SC (FIG. 2e-h, v, Supplementary Video 2, FIG. 8e-h). By P2, the sprouting ECs had coalesced to form a rudimentary SC and connections to the CCs were lost. The transscleral vessels that remained attached to the rudimentary SC represent the future AVs (FIG. 2i-l, w, Supplementary Video 3, FIG. 8i-l). At P2, cells of the rudimentary SC furthest away from the two long posterior ciliary arteries were observed to express low levels of Prox1 (FIG. 2j, indicated by *). By P4, the cells expressed Prox1 throughout the canal (FIG. 2n, indicated by *), weak VEGFR3 expression was detected and the canal had undergone further luminalization; fragments of it could be detected in the H&E stained sections (FIG. 2m-p, x, Supplementary Video 4, FIG. 8m-p). By P7, the SC had grown in width and expressed high levels of VEGFR-3, resembling the mature SC and appearing as a uniform canal in H&E stained sections (FIG. 2q-t, y, Supplementary Video 5, FIG. 8q-t). Prox1 and VEGFR-3 expression levels were maintained throughout adulthood (FIG. 1b-c), and Prox1 promoter activity was detected even at 18 months of age (FIG. 7m). The related VEGFR-2 was detected in the thick sections at all stages of SC development, in the CCs, and the ES vasculature (FIG. 8).

The Lymphangiogenic Growth Factor VEGF-C is Critical for SC Development.

The close resemblance between the development of the SC and the lymph sacs led us to hypothesize that the lymphangiogenic growth factor VEGF-C plays a critical role also in SC development. Vegfc^{-/-} mouse embryos are characterized by a failure to form the initial LEO sprouts^{15,16}. However, these mice cannot be studied postnatally due to embryonic lethality. We therefore analyzed Vegfc heterozygous (Vegfc^{+/LacZ}) mice¹⁶, conditionally Vegfc deleted mice (Vegfc^{flox/flox}; R26-iCreER^{T2})(Ref. ²⁷, Harri Nurmi, manuscript in preparation), VEGF-D knockout mice (VEGF-D^{-/-})²³, and transgenic mice expressing soluble VEGFR-3, which blocks VEGF-C and VEGF-D activity (K14-VEGFR-3(1-3)-Ig)²¹) or a corresponding protein that does not trap these factors (K14-VEGFR-3(4-7)-Ig)²².

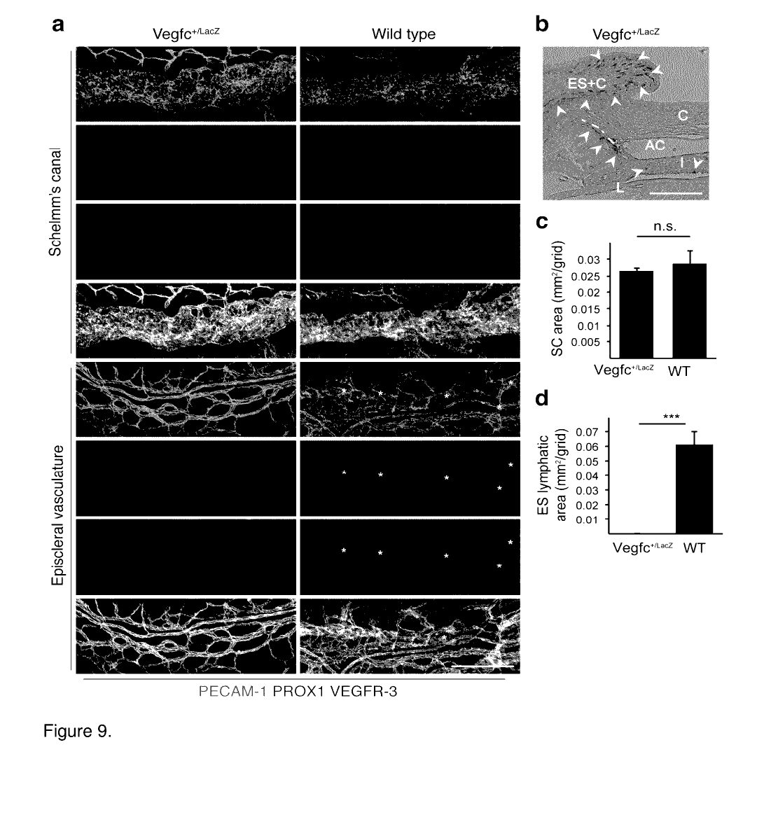

During development, VEGF-C is expressed predominantly in regions where lymphatic vessels develop¹⁶. In the Vegfc heterozygous mice, in which the LacZ gene encoding β-galactosidase has been inserted into the Vegfc locus (Vegfc^{+/LacZ}), X-gal staining revealed prominent VEGF-C expression adjacent to the SC. However, despite the total lack of ES lymphatic vasculature in the Vegfc^{+/LacZ} pups, the SC appeared normal in comparison with the wild type littermates (FIG. 9).

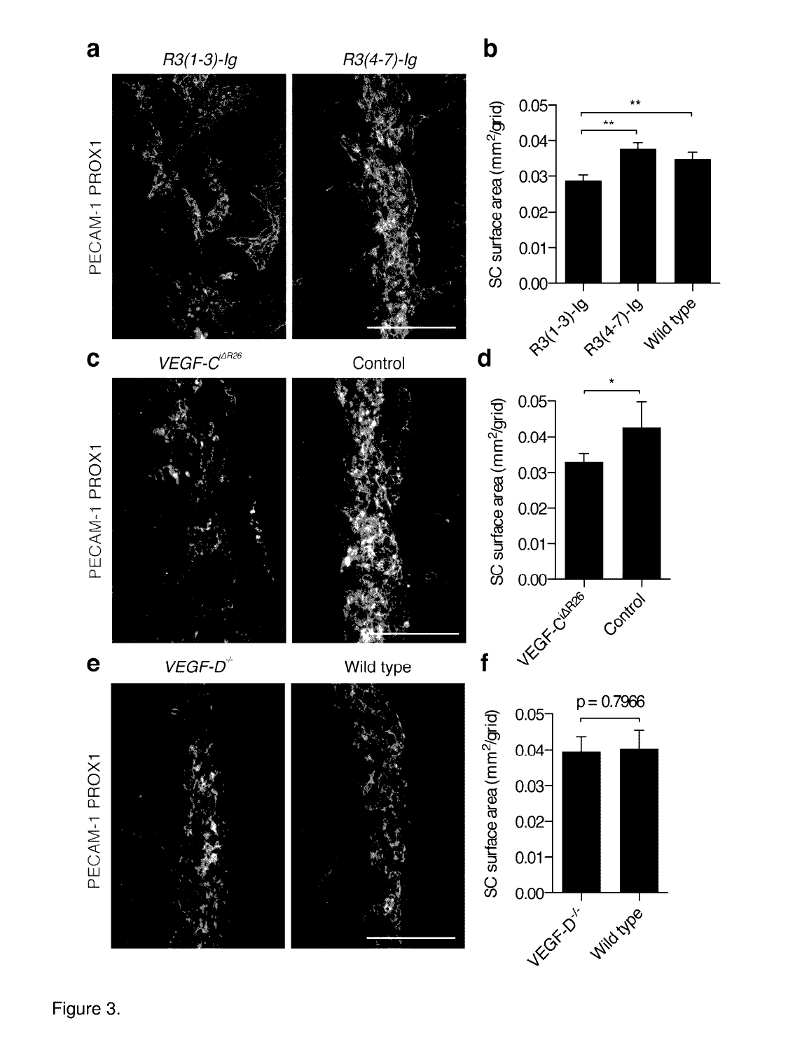

When SC morphology was assessed at P7 in the transgenic mice that express the soluble VEGFR-3 fusion proteins, the K14-VEGFR-3(1-3)-Ig mice were distinguished from their wild-type littermates and the K14-VEGFR3(4-7)-Ig control mice by their markedly hypoplastic SC characterized by lacunae that were disconnected from each other, and by the reduction of the SC surface area (FIG. 3a-b). Both VEGF-C and VEGF-D are neutralized by the VEGFR-3(1-3)-Ig transgene-encoded protein. To dissect which of these factors is required for SC development, we analyzed Vegfd^{-/-} mice and mice conditionally deleted of Vegfc by using the Rosa26-CreER^{T2} allele that globally expresses a tamoxifen-activated Cre recombinase. When the SC morphology was assessed after daily 4-hydroxytamoxifen (4-OHT) injections (from P1 to P5) in the VEGF-C deleted mice at P7, abnormal hypoplastic SC morphology and a reduction of SC surface area was observed, reminiscent of the K14-VEGFR-3(1-3)-Ig mice (FIG. 3c-d). In contrast, no defect could be detected in SC development in the Vegfd^{-/-} pups (FIG. 3e-d).

The Lymphangiogenic Receptor VEGFR-3 is Critical for SC Development.

VEGFR-3 tyrosine kinase activity is essential for lymphatic vessel growth³⁸. VEGFR-3 is activated by VEGF-C and VEGF-D, and VEGFR-3 mutations in both mice and in patients with Milroy disease result in defective development of the lymphatic vasculature, resulting in lymphedema³⁹. The role of VEGFR-3 signaling in SC development was assessed in Chy mice²⁴, a genetic model of Milroy disease with a heterozygous kinase-inactivating point mutation in the VEGFR-3 tyrosine kinase domain, in mice administered with the VEGFR-2 and VEGFR-3 blocking monoclonal antibodies DC101³⁶ and mF4-31C³⁶, and in mice in which Vegfr3 or Vegfr2 was conditionally deleted specifically in the SC endothelium (Vegfr3^{flox/flox}; Prox1-CreER^{T2} and Vegfr2^{flox/flox}; Prox1-CreER^{T2})^{25,26,28}.

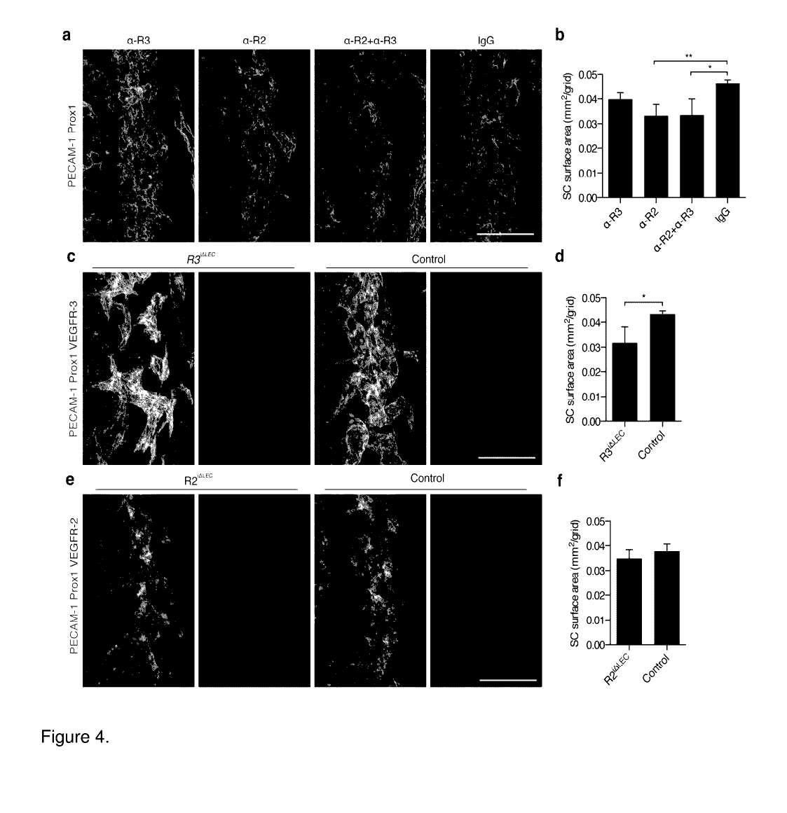

In the Chy mice, lack of ES lymphatic vasculature was observed as in the Vegfc heterozygous mice. However, as in the Vegfc^{+/LacZ} mice, no defects were observed in the SC by immunofluorescence at P12 (FIG. 10). When VEGFR-2, VEGFR-3 or the combination of VEGFR-2 and VEGFR-3 blocking antibodies was administered daily from P0 to P6, SC hypoplasia was detected most significantly in the anti-VEGFR-2 and anti-VEGFR-2/3 treated mice at P7, whereas blocking VEGFR-3 did not lead to a statistically significant reduction in SC area. In addition, no additive effects were detected when both VEGFR-3 and VEGFR-2 antibodies were used (FIG. 4a-b). Taken together, these findings indicate that the early stages of SC development involve VEGFR-2 signaling. The minimal phenotype observed with the blocking antibodies was found to result from reduced bioavailability of the antibodies in the eye after the SC transitions into a blind-ended tube and the subsequent development of the blood-eye-barrier at P3-4 (FIG. 8u-x).

The functional importance of VEGFR-3 and VEGFR-2 in SC development was further examined with SC specific deletion of Vegfr3 and Vegfr2. Induction of Cre activity in Vegfr3^{flox/flox}; Prox1-iCreER^{T2} mice by daily 4-OHT injections from P1 to P5 resulted in a markedly hypoplastic SC with reduced surface area at P7 when compared to Vegfr3^{flox/flox} control littermates, indicating a critical role of VEGFR-3 in SC development. Vegfr3 deleted mice were characterized by SC lacunae that failed to connect with each other similar to the K14-VEGF-3-Ig mice and in mice conditionally deleted of Vegfc. No residual VEGFR-3 staining was detected in these mice (FIG. 4c-d). No SC defects could be detected in Vegfr2^{flox/flox}; Prox1-CreERT2 mice as compared to Vegfr2^{flox/flox} littermate control, which may result from incomplete gene deletion (FIG. 4e-f).

VEGF-C Administration Induces Sprouting, Proliferation and Migration of the SC ECs Toward VEGF-C Gradients in Adults.

VEGF-C has been shown to induce sprouting, proliferation, migration and survival of LECs, both in vitro and in vivo in adults. Therapeutic lymphangiogenesis with viral vectors encoding VEGF-C is being developed for clinical use in the regeneration of lymphatic vessels and treatment of lymphedema^{31,33,40-42.} The role of VEGF-C/VEGFR-3 signaling in SC development led us to hypothesize that VEGF-C could be used for the therapeutic manipulation of the SC in order to facilitate AH outflow in the treatment of glaucoma. To do this, we first analyzed the effects of VEGF-C overexpression in the anterior segment of the eye with adenovirus or adeno-associated virus (AAV) vectors.

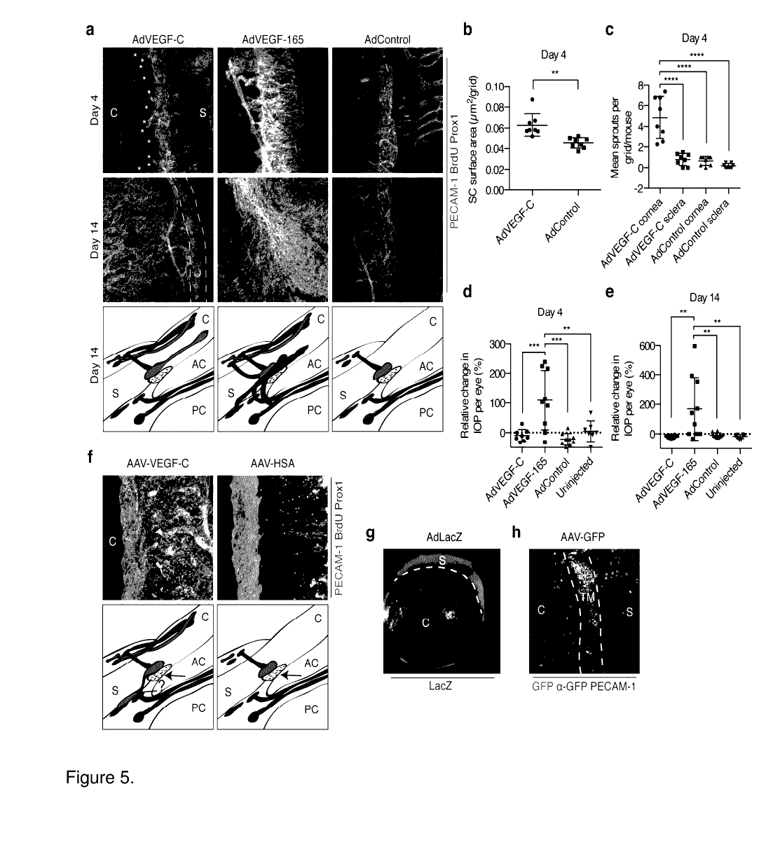

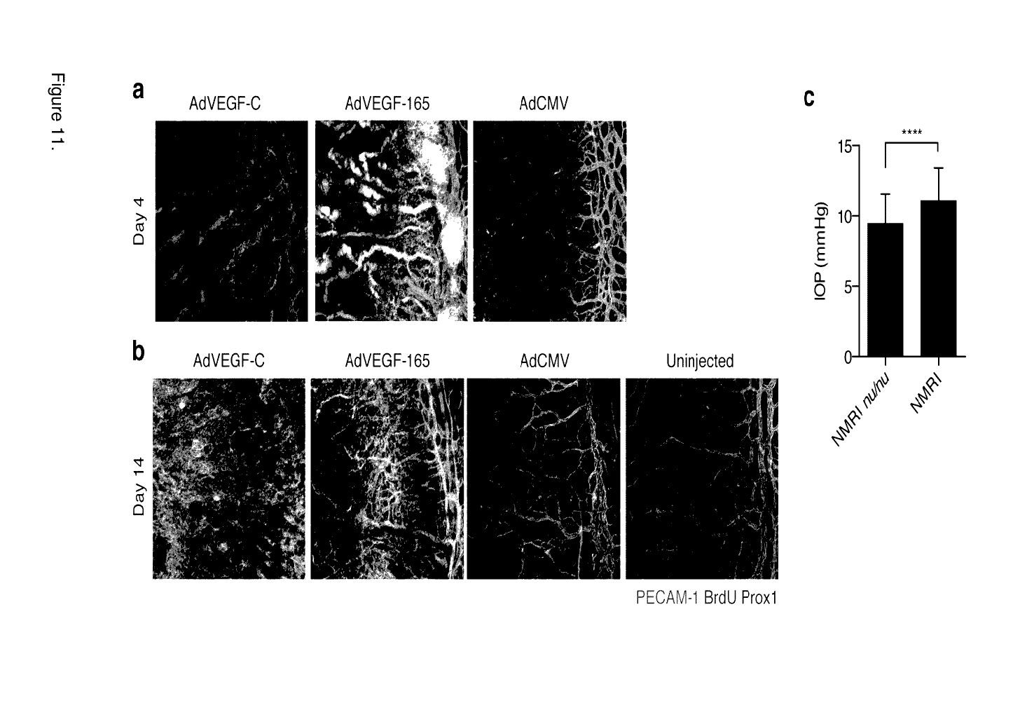

Adenoviral vectors provide transient transgene expression with highest levels within days after injection⁴³. Adenoviruses encoding VEGF-C (AdVEGF-C) or VEGF165 (AdVEGF), or an “empty” control vector (AdControl), were injected into the anterior chamber of NMRI nu/nu mice. The eyes were analyzed at day 4 and day 14. To assess effects on aqueous outflow facility, IOP measurements were performed before injection and before sacrifice. While treatment with AdVEGF was associated with a marked increase in intraocular pressure, essentially resulting in neovascular glaucoma, the AdVEGF-C treated eyes had normal IOP comparable to Ad control injected and uninjected eyes (FIG. 2b). The effects of VEGF-C and VEGF overexpression on the SC endothelium were studied in whole mount eyes stained for PECAM-1 and Prox1. To identify proliferating ECs, the mice received an injection of bromodeoxyuridine (BrdU) 2 h prior to sacrifice and the BrdU incorporated to nuclear DNA was stained. At day 4, marked sprouting and proliferation of the SC endothelium was detected in the VEGF-C treated eyes. Sprouts from the SC endothelium extended almost exclusively towards the inner surface of the cornea (FIG. 2a-b, c, sprouts indicated with an asterisk). Surprisingly, at 2 weeks, large areas of the cornea were filled with Prox1-positive SC endothelium connected to the original circular SC (original SC indicated by dashed line), indicating that VEGF-C has highly specific effects on the SC endothelium. To explain the corneal involvement, AdLacZ reporter was used. Beta-galactosidase staining revealed effective transfection of the cornea. (FIG. 5g). AdVEGF treatment entirely obliterated the aqueous outflow system, and by day 14, only a sheet of ECs surrounding the limbus could be detected. (FIG. 5a). While corneal vessels were detected even in uninjected NMRI nu/nu mice, angiogenesis and lymphangiogenesis were observed in both AdVEGF and AdVEGF-C injected eyes. (FIG. 11a-b).

To study the effects of long-term overexpression of VEGF-C, AAV vectors encoding VEGF-C, VEGF or human serum albumin (HSA) were injected into the anterior chamber of NMRI nu/nu mice and the eyes were analyzed 6 weeks after transduction. Surprisingly, AAV-VEGF-C injection resulted in the extension of Prox1-positive SC outpocketing toward the sclera as opposed to the cornea in the AdVEGF-C injected eyes (FIG. 2L-O). AAV-VEGF injected mice displayed obliteration of the eye associated with massive increase in intraocular pressure, forcing an early sacrifice after 2 weeks. These experiments suggested that VEGF-C can be used for the therapeutic manipulation of the SC whereas VEGF essentially destroys the AH drainage system, which is likely to be the pathophysiological mechanism of neovascular glaucoma.

A Single Injection of Recombinant VEGF-C Induces Sprouting, Proliferation and Enlargement of the SC ECs and a Sustained Decrease in Intraocular Pressure.

In NMRI nu/nu mice, IOP is substantially lower than in wild-type NMRI mice (FIG. 11c) and in other mouse strains⁴⁴. This led us to speculate that no IOP lowering effect could be detected in the NMRI nu/nu mice. In order to study the potential of VEGF-C in facilitating aqueous outflow, we chose to study wild-type NMRI mice and use recombinant VEGF-C in order to avoid potential detrimental effects of sustained protein production via viral vectors, such as corneal neovascularization. First, to provide proof-of-principle that recombinant VEGF-C induces growth of the SC, recombinant VEGF-C (rVEGF-C), VEGF-165 (rVEGF) or HSA was injected into the anterior chamber on three consecutive days. On analysis at day 4, VEGF-C induced proliferation and sprouting of the SC ECs preferentially toward the sclera while VEGF-165 obliterated the vascular aqueous outflow system (FIG. 2a). While rVEGF induced massive corneal angiogenesis, rVEGF-C induced only mild corneal lymphangiogenesis and some angiogenesis (FIG. 11). In these mice, no reliable IOP data could be obtained due to the tree consecutive injections (data not shown). To overcome corneal neovascularization and in order to study the effects of recombinant VEGF-C on aqueous outflow, we injected a single dose of 4.8 μg of rVEGF-C or recombiant mouse serum albumin (rMSA) into the anterior chamber and analyzed the effect on IOP. Surprisingly, a single injection of rVEGF-C resulted in a sustained decrease in IOP detected at days 9 (−30.03%±5.599 vs. −6.684%±3.597), 11 (−28.52%±5.034 vs. −5.198±3.106) and 14 (−27.86%±5.117 vs. −1.705%±4.625) after injection. Macroscopically, all rVEGF-C injected eyes appeared normal. Furthermore, no pathology could be detected in H&E staining of rVEGF-C injected eyes at 2 weeks. In whole mount immunofluorescence, rVEGF-C treatment induced mild growth of the SC toward the sclera. Moreover, rVEGF-C did not induce any corneal neovascularization. These results indicate that a single injection of low-dose rVEGF-C safely induces growth of the SC that is associated with a substantially higher and sustained decrease in IOP (−28 to −30%) compared to the transient IOP lowering effects achievable with current eyedrop therapies achievable in normotensive mice^{18,19}.

### REFERENCE LIST

1. Kwon, Y. H., Fingert, J. H., Kuehn, M. H. & Alward, W. L. M. Primary Open-Angle Glaucoma. N. Engl. J. Med. 360, 1113-1124 (2009).
2. Coleman, A. L. Glaucoma. Lancet 354, 1803-1810 (1999).
3. Resnikoff, S. et al. Global data on visual impairment in the year 2002. Bull. World Health Organ. 82, 844-851 (2004).
4. Quigley, H. A., Broman, A. T. & Broman, A. T. The number of people with glaucoma worldwide in 2010 and 2020. Br J Ophthalmol 90, 262-267 (2006).
5. Ramos, R. F., Hoying, J. B., Witte, M. H. & Daniel Stamer, W. Schlemm??s Canal Endothelia, Lymphatic, or Blood Vasculature? J. Glaucoma 16, 391-405 (2007).
6. Fechtner, R. D. & Weinreb, R. N. Mechanisms of optic nerve damage in primary open angle glaucoma. Surv Ophthalmol 39, 23-42 (1994).
7. Flammer, J. et al. The impact of ocular blood flow in glaucoma. Prog Retin Eye Res 21, 359-393 (2002).
8. The Ocular Hypertension Treatment Study: a randomized trial determines that topical ocular hypotensive medication delays or prevents the onset of primary open-angle glaucoma. 120, 701-13-discussion 829-30 (2002).
9. Heijl, A. et al. Reduction of intraocular pressure and glaucoma progression: results from the Early Manifest Glaucoma Trial. Arch. Ophthalmol. 120, 1268-1279 (2002).
10.
11. Schlemms Canal in OCT Images in Glaucoma Patients and Healthy Subjects. 04, (2013).
12. Libby, R. T., Libby, R. T., Gonzalez, F. J. & John, S. W. M. Modification of Ocular Defects in Mouse Developmental Glaucoma Models by Tyrosinase. Science 299, 1578-1581 (2003).
13. Perry, L. P., Jakobiec, F. A., Zakka, F. R. & Walton, D. S. Newborn primary congenital glaucoma: Histopathologic features of the anterior chamber filtration angle. Journal of American Association for Pediatric Ophthalmology and Strabismus 16, 565-568 (2012).
14. Smith, R. S. et al. Haploinsufficiency of the transcription factors FOXC1 and FOXC2 results in aberrant ocular development. Hum. Mol. Genet. 9, 1021-1032 (2000).
15. Hägerling, R. et al. A novel multistep mechanism for initial lymphangiogenesis in mouse embryos based on ultramicroscopy. EMBO J. 32, 629-644 (2013).
16. Karkkainen, M. J. et al. Vascular endothelial growth factor C is required for sprouting of the first lymphatic vessels from embryonic veins. Nat Immunol 5, 74-80 (2003).
17. Oliver, G., Oliver, G., Srinivasan, R. S. & Srinivasan, R. S. Endothelial cell plasticity: how to become and remain a lymphatic endothelial cell.
Development 137, 363-372 (2010).
18. Johnson, T. V., Fan, S. & Toris, C. B. Rebound Tonometry in Conscious, Conditioned Mice Avoids the Acute and Profound Effects of Anesthesia on Intraocular Pressure. Journal of Ocular Pharmacology and Therapeutics 24, 175-185 (2008).
19. Reduction of intraocular pressure in mouse eyes treated with latanoprost. 43, 146-150 (2002).
20. Petrova, T. V. et al. Transcription Factor PROX1 Induces Colon Cancer Progression by Promoting the Transition from Benign to Highly Dysplastic Phenotype. Cancer Cell 13, 407-419 (2008).
21. Makinen, T. et al. Nat. Med. 7, 199-205 (2001).
22. VEGF-C and VEGF-D Blockade Inhibits Inflammatory Skin Carcinogenesis. 73, 4212-4221 (2013).
23. Baldwin, M. E. et al. Vascular Endothelial Growth Factor D Is Dispensable for Development of the Lymphatic System. Mol. Cell. Biol. 25, 2441-2449 (2005).
24. Karkkainen, M. J. et al. A model for gene therapy of human hereditary lymphedema. Proceedings of the National Academy of Sciences 98, 12677-12682 (2001).
25. Haiko, P. et al. Deletion of Vascular Endothelial Growth Factor C (VEGF-C) and VEGF-D Is Not Equivalent to VEGF Receptor 3 Deletion in Mouse Embryos. Mol. Cell. Biol. 28, 4843-4850 (2008).
26. Albuquerque, R. J. C. et al. Alternatively spliced vascular endothelial growth factor receptor-2 is an essential endogenous inhibitor of lymphatic vessel growth. Nat. Med. 15, 1023-1030 (2009).
27. Vooijs, M., Jonkers, J., Vooijs, M. & Berns, A. A highly efficient ligand-regulated Cre recombinase mouse line shows that LoxPrecombination is position dependent. EMBO reports 2, 292-297 (2001).
28. Bazigou, E. et al. Genes regulating lymphangiogenesis control venous valve formation and maintenance in mice. J. Clin. Invest. 121, 2984-2992 (2011).
29. Hägerling, R., Pollmann, C., Kremer, L., Andresen, V. & Kiefer, F. Intravital two-photon microscopy of lymphatic vessel development and function using a transgenic Prox1 promoter-directed mOrange2 reporter mouse. Biochem. Soc. Trans. 39, 1674-1681 (2011).
30. Madisen, L. et al. A robust and high-throughput Cre reporting and characterization system for the whole mouse brain. Nat Neurosci 13, 133-140 (2009).
31. Tammela, T. et al. Angiopoietin-1 promotes lymphatic sprouting and hyperplasia. Blood 105, 4642-4648 (2005).
32. Anisimov, A. et al. Activated Forms of VEGF-C and VEGF-D Provide Improved Vascular Function in Skeletal Muscle. Circ. Res. 104, 1302-1312 (2009).
33. Tammela, T. et al. Therapeutic differentiation and maturation of lymphatic vessels after lymph node dissection and transplantation. Nat. Med. 13, 1458-1466 (2007).
34. Enholm, B. et al. Adenoviral expression of vascular endothelial growth factor-C induces lymphangiogenesis in the skin. Circ. Res. 88, 623-629 (2001).
35. Saeki, T., Aihara, M., Ohashi, M. & Araie, M. The Efficacy of TonoLab in
Detecting Physiological and Pharmacological Changes of Mouse Intraocular Pressure—Comparison with TonoPen and Microneedle Manometery. Curr Eye Res 33, 247-252 (2008).
36. Tammela, T. et al. Blocking VEGFR-3 suppresses angiogenic sprouting and vascular network formation. Nature 454, 656-660 (2008).
37. Hamanaka, T. et al. Aspects of the development of Schlemm's canal. Exp. Eye Res. 55, 479-488 (1992).
38. Tammela, T. & Alitalo, K. Lymphangiogenesis: Molecular Mechanisms and Future Promise. Cell 140, 460-476 (2010).
39. Alitalo, K. The lymphatic vasculature in disease. Nat. Med. 17, 1371-1380 (2011).
40. Lymph node transfer and perinodal lymphatic growth factor treatment for lymphedema. 257, 961-967 (2013).
41. Lähteenvuo, M. et al. Growth factor therapy and autologous lymph node transfer in lymphedema. Circulation 123, 620 (2011).
42. Hartiala, P. & Saaristo, A. M. Growth factor therapy and autologous lymph node transfer in lymphedema. Trends Cardiovasc. Med. 20, 249-253 (2010).
43. Intraocular adenoviral vector-mediated gene transfer in proliferative retinopathies. 43, 1610-1615 (2002).
44. John, S. W., Hagaman, J. R., MacTaggart, T. E., Peng, L. & Smithes, O. Intraocular pressure in inbred mouse strains. Invest. Ophthalmol. Vis. Sci. 38, 249-253 (1997).

1. A VEGFR-3 activating ligand or a composition comprising a VEGFR-3 activating ligand for use in treating ocular hypertension or glaucoma in a subject, wherein the VEGFR-3 activating ligand is VEGF-C.

2. A method of treating ocular hypertension or glaucoma by administering to a subject in need thereof a VEGFR-3 activating ligand or a composition comprising a VEGFR-3 activating ligand, wherein the VEGFR-3 activating ligand is VEGF-C.

3. The VEGFR-3 activating ligand or the composition for use according to claim 1, wherein VEGF-C is a human VEGF-C.

4. The VEGFR-3 activating ligand or the composition for use of claim 1, wherein VEGF-C is in a form of a fusion protein.

5. The composition for use of claim 1, wherein the composition further comprises a pharmaceutically acceptable vehicle.

6. The composition for use of claim 1, wherein the composition further comprises CCBE1 and/or VEGF-D.

7. The composition for use of claim 1, wherein VEGF-C is the only therapeutically effective agent.

8. The composition for use of claim 1, wherein the composition further comprises other therapeutically effective agents.

9. The VEGFR-3 activating ligand or the composition for use of claim 1, wherein the VEGFR-3 activating ligand or the composition is used concurrently with other therapeutic agents or therapeutic methods.

10. The VEGFR-3 activating ligand or the composition for use according to claim 9, wherein the therapeutic method is a surgical method.

11. The VEGFR-3 activating ligand or the composition for use of claim 1, wherein the subject is a human or an animal.

12. The VEGFR-3 activating ligand or the composition for use of claim 1, wherein glaucoma is selected from the group consisting of primary glaucoma and its variants, developmental glaucoma, secondary glaucoma and absolute glaucoma.

13. The method of claim 2, wherein VEGF-C is a human VEGF-C.
